SEQUENCE LISTING (9 sequences)

SEQ ID NO: 1 - DNA, 33 bp, T. thermophila
aattcgtaatacgactcactataggttctatag

SEQ ID NO: 2 - DNA, 25 bp, T. thermophila
agaacctatagtgagtcgtattacg

SEQ ID NO: 3 - DNA, 28 bp, T. thermophila
tatagcggttagtactggggactctaaa

SEQ ID NO: 4 - DNA, 28 bp, T. thermophila
gagtccccagtactaaccgctatactat

SEQ ID NO: 5 - DNA, 18 bp, T. thermophila
tcccttgacctgggttcg

SEQ ID NO: 6 - DNA, 18 bp, T. thermophila
cccaggtcaagggattta

SEQ ID NO: 7 - DNA, 32 bp, T. thermophila
aatcccagtaggaccgccatgagacccatccg

SEQ ID NO: 8 - DNA, 39 bp, T. thermophila
gatccggatgggtctcatggcggtcctactgggattcga

SEQ ID NO: 9 - protein, 1159 aa, HERG ion channel
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

(19) United States
(12) Patent Application Publication
(10) Pub. No.: US 2006/0084102 A1
(43) Pub. Date: Apr. 20, 2006

(54) Methods for determining precise HERG interactions by mutagenesis

(75) Inventors: Dennis A. Dougherty, South Pasadena, CA (US); Henry A. Lester, South Pasadena, CA (US); Mark W. Nowak, Pasadena, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neurion Pharmaceuticals, Inc., Pasadena, CA (US)

(21) Appl. No.: 11/242,822

(22) Filed: Oct. 3, 2005

Related U.S. Application Data
(63) Continuation-in-part of application No. 10/957,116, filed on Oct. 1, 2004.
Application No. 10/957,116 is a continuation-in-part of application No. 10/444,058, filed on May 23, 2003.
(60) Provisional application No. 60/615,173, filed on Oct. 1, 2004.
(60) Provisional application No. 60/382,571, filed on May 24, 2002.
(60) Provisional application No. 60/454,338, filed on Mar. 14, 2003.

(51) Int. Cl. C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

The present invention discloses methods of determining highly precise interactions between the HERG ion channel and various compounds. The methods of the present invention utilize modified HERG ion channels combined with heterologous in vivo expression in Xenopus oocytes and mammalian cells.

### CROSS-REFERENCES TO RELATED APPLICATIONS

This application claims priority to U.S. Provisional Patent Application 60/615,173, filed Oct. 1, 2004 entitled “Methods of Determining Precise HERG Interactions by Mutagenesis” and is further related to U.S. patent application Ser. No. 10/444,058, filed May 23, 2003, entitled “Methods of Determining Precise HERG Interactions and Altering Compounds Based on Said Interactions”, and the continuation-in-part U.S. patent application Ser. No. 10/957,116, filed Oct. 1, 2004, of the same title as well as U.S. Provisional Patent Application 60/382,571, filed May 24, 2002, and 60/454,338, filed Mar. 14, 2003, both entitled “Methods of Determining Precise HERG Interactions and Designing Compounds Based on Said Interactions”. All five applications are hereby incorporated in their entireties as if fully set forth.

### FIELD OF THE INVENTION

The present invention generally relates to methods of obtaining high-precision structural and functional information on the membrane protein ion channel HERG. The present invention more specifically relates to mutagenesis methods to produce modified HERG in vivo and via heterologous expression. These methods permit determination of the details of HERG binding to individual compounds to a very high specificity. The binding to HERG by many different classes of molecules can lead to potentially fatal cardiac side effects. The present invention also relates to methods for the discovery and design of safer and more selective compounds without HERG activity.

### BACKGROUND OF THE INVENTION

Voltage-gated potassium channels are key determinants of normal cellular activity, but can contribute to disease and, consequently, are increasingly recognized as potential therapeutic targets. Changes in the properties of potassium channels and even the types expressed have been linked to several cardiac and neurological diseases. Nerbonne (1998) J Neurobiol. 37:37-59.

The human ether-a-go-go related gene HERG encodes the HERG K⁺ channel, which is one of the ion channels responsible for generating the cardiac action potential. HERG encodes an inwardly-rectifying potassium channel that plays an important role in repolarization of the cardiac action potential. Inward rectification of HERG channels results from rapid and voltage-dependent inactivation gating, combined with very slow activation gating.

HERG was originally cloned from human hippocampus by Warmke et al. (1994) Proc. Natl. Acad. Sci USA 91:3438-3442, and is strongly expressed in the heart. The hydropathy plot for the HERG protein suggests that this channel resembles the Shaker potassium channel; both have a six transmembrane region subunit structure with a highly charged fourth transmembrane segment. Despite this similarity, HERG channels behave very differently from Shaker channels: HERG behaves like an inward rectifier rather than an outward rectifier. Sanguinetti et al. (1995) Cell 81:299-307. This anomalous behavior is due to the unusual kinetics of HERG gating. During depolarization, HERG channels slowly activate and then rapidly inactivate, resulting in little outward current. During subsequent hyperpolarization, channels recover rapidly from inactivation but deactivate slowly, resulting in a large inward current.

Long QT syndromes (LQTs) have in common an abnormality of cardiac muscle repolarization that predisposes affected individuals to a ventricular arrhythmia that can degenerate into ventricular fibrillation and cause sudden death. The HERG ion channel has been linked to QT interval prolongation and sudden death. Mutations in the HERG channel gene cause one form of the inherited long QT syndromes (LQT2). QT interval prolongation can also be caused by non-genetic, or extrinsic causes. In recent years, several prescription drugs have been shown to be responsible for this QT interval prolongation, and linked to HERG activity. Drugs such as Seldane, Propulsid, Hismanal, and others have been removed from the market because of their potential cardiac side effects and suspected HERG activity. Additionally, many promising drugs in clinical trials and pre-clinical compounds have been removed from the development pathway because of activity at the HERG ion channel. This has led to literally billions of dollars of lost revenues and wasted development costs.

Small moleule binding to HERG is an area of increasing focus and frustration for the pharmaceutical industry. The FDA requires that companies demonstrate safety for new drugs. Most pharmaceutical companies have detailed in vitro and in vivo pre-clinical tests to screen for potentially hazardous compounds that prolong the QT interval on ECG readings (“ICH Guideline on Safety Pharmacology Studies for Human Pharmaceuticals” (ICH S7A), Feb. 7, 2002). Therefore, methods of determining this unexpected activity are highly desirable to the pharmaceutical industry and for ensuring patient safety. Methods of nonsense codon suppression have been used to probe structure-function relationships in receptor binding sites of other ion channels. Nowak et al. (1995) Science 268:439. The method of combining site-directed mutagenesis and heterologous expression was instrumental in elucidating the functional relationships of the nicotinic receptor with its agonists and antagonists. Id. Application of these methods to the HERG system may help elucidate the interactions between small molecules and HERG and possibly mitigate the binding interactions that lead to prolonged QT intervals.

Current small molecules-HERG screens reveal information about the existence and strength of HERG binding, but does not give precise details on the nature and location of the binding, nor indications of about how one could make subtle modifications to compounds in order to remove HERG binding.

Citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

The instant invention is based upon the recognition that modified (mutagenized) forms of the HERG ion channel may be used to advance the determination of interactions between a molecule or compound and an unmodified form of the HERG ion channel. The present invention will not only provide information on whether a molecule or compound binds to HERG, but also details the specific interactions involved in drug binding. Such details provide the opportunity for high-precision modifications to the molecule or compound, to reduce or eliminate undesirable effects on HERG activity. The invention thus permits the identification and continued development of drug classes that would otherwise be dropped because of undesirable HERG activity.

Thus in a first aspect, the invention provides for modified forms of the HERG ion channel wherein an amino acid residue present in HERG, as found in naturally occurring sources, is replaced with a non-native amino acid residue. The non-native amino acid may be a naturally occurring amino acid or an unnatural amino acid as described herein and known in the art.

The modified forms of the HERG ion channel are produced by expression in an appropriate system, preferably a cell based system wherein HERG can be expressed in a functional form within the membranes of the cell. The cells are preferably eukaryotic cells, and the expression is preferably within the cell surface membranes of a cell such that binding of a molecule or compound to the expressed HERG on the exterior of the cell can be readily accomplished. Preferred cells of the invention are those suitable for assessment of HERG activity in the presence and absence of a molecule or compound to be assayed for HERG binding and/or modulation.

The binding of a molecule or compound to HERG can be used to determine the nature of the interactions between the molecule or compound and HERG. These interactions include those on a submolecular, but greater than atomic, level such that interactions between a molecule or compound and particular amino acid side chains of HERG can be studied. These regions or sites of interaction with HERG by a molecule or compound represent valuable information provided by practice of the invention. Thus in a second aspect, the invention provides methods of determining precise compound interactions with the HERG ion channel. These methods utilized the modified HERG ion channels of the invention to assess the relationship between the structure of a molecule or compound and any HERG binding function present in the molecule or compound. The invention thus permits the detailed determination of the nature and form of interactions between HERG and a molecule or compound which binds HERG.

The invention provides methods of determining the sites of interaction, as well as the nature of the interaction at such sites (and so both “where” and “how” of the interactions), between a molecule or compound and HERG. Such methods include those comprising the use of a modified HERG ion channel to identify the sites of interaction with a molecule or compound that has undesirable HERG activity upon binding of the molecule or compound to HERG. This ability to determine the sites of interaction, which are sites of potential antagonist or agonist activity in the HERG ion channel, may be used to modify the molecule or compound such that undesirable HERG activity is reduced or eliminated. Thus the invention provides a method for the determination of sites of interaction between a molecule or compound and HERG by contacting a modified HERG with the molecule or compound. The methods may also be used to determine the nature of the interactions between a molecule or compound and a modified HERG. These methods may optionally include the subsequent use of the information to modify a molecule or compound. Thus a molecule or compound which binds to result in undesirable HERG activity, and so to lead to undesirable effects including, but not limited to, cardiac arrhythmia, ventricular fibrillation, QT interval prolongation, and sudden death, can be identified and altered to attenuate such negative effects.

The invention also provides for the use of the above in a method of determining where to replace amino acids in HERG, said method comprising: a) determining site(s) of potential antagonist or agonist interaction with the HERG ion channel; b) modifying the HERG site(s) determined in (a) to include a non-native amino acid residue in such a site; and c) determining binding interactions of a molecule or compound of interest with the modified HERG ion channel.

The invention also provides a systematic method of determining the nature of the interaction between HERG and a molecule or compound by comparison of the binding of unmodified and modified HERG to the molecule or compound. Such methods include those comprising: a) incorporating a non-native amino acid into the binding and/or regulatory site(s) of HERG to produce a modified or altered HERG; b) measuring the ability of the molecule or compound to bind to the modified or altered HERG; and c) comparing the results of step (b) to the same compound's ability to bind to an unmodified or unaltered HERG. The comparison may be performed by comparison of the nature or strength of interactions between the molecule or compound and the modified and unmodified HERG. Additionally, the invention provides for comparisons, as described above, of the binding of a molecule or compound to one modified HERG relative to another modified HERG. The results of any of these comparisons, or combinations thereof, may be used to modify the molecule or compound such that undesirable HERG activity is reduced or eliminated in the presence of the molecule or compound.

The invention also provides methods to assay a molecule or compound for cardiac toxicity or other unwanted effects on HERG activity or resulting therefrom. Non-limiting examples of effects resulting from unwanted HERG activity include cardiac arrhythmia, ventricular fibrillation, QT interval prolongation, and sudden death. Such assays utilize the methods to determine and identify binding as well as the nature of the binding between HERG and a molecule or compound as disclosed herein.

It is yet a further aspect of the invention to provide a systematic method of screening compounds which cause cardiac toxicity to determine the location and form of the interactions between the compounds and HERG which give rise to the toxicity. The resulting information may be used to modify existing compounds to reduce or eliminate their HERG mediated toxicity or to design new compounds with reduced or eliminated HERG mediated toxicity. In one form, the method comprises a) identifying a plurality of compounds that prolong QT interval on ECG readings; b) determining details of the nature and location of HERG binding of each of said compounds by use of modified and unmodified HERG as disclosed herein; and c) determining how each compound is causing said toxicity by evaluating the nature and location of the binding of each compound to HERG.

It is another aspect of the invention to provide a receptophore model, which provides a 3-dimensional picture of compounds contact points at the HERG channel binding sites based upon analysis using the modified HERG of the invention.

It is also an aspect of the invention to provide a method of altering a compound so that it does not interact with HERG comprising: a) determining the nature of the compound's interaction with HERG or a modified HERG; b) analyzing how and where the compound interacts with HERG or the modified HERG; based on the analysis in step (b), and c) chemically modifying the compound to avoid HERG interaction.

The invention also provides for the generation of a dataset of information for individual compounds and agents describing the activity of each with modified and unmodified HERG channels modified with a non-native amino acid. The information reflects the specific binding interactions, or lack thereof, that contribute to the binding of a compound or agent to HERG, particularly at key amino acid residues. This information provides the ability to engineer drug compounds and agents to avoid interactions with key HERG amino acid side chains and thus avoid or eliminate cardiac liability such as, but not limited to, cardiac arrhythmias, cardiac dysfunctions, and/or sudden death. The invention may thus also be used to optimize lead drug compounds or agents to reduce or avoid undesirable interactions with HERG.

### BRIEF DESCRIPTION OF THE DRAWINGS

FIG. 1 is a scheme for incorporating unnatural amino acids into proteins expressed in Xenopus oocytes.

FIG. 2 is a plot of log[EC₅₀/EC_{50(WT)}] vs. cation-π binding ability at α-Trp149 of the nicotinic acetylcholine receptor for the wild type Trp and the fluorinated Trp derivatives 5-F-Trp, 5,7-F₂-Trp, 5,6,7-F₃-Trp, and 4,5,6,7-F₄-Trp.

FIG. 3 is a schematic illustrating how a given molecule, with astemizole as exemplification, might be postulated to interact with HERG. Hydrogen bonding might be thought to occur via positions Thr623, Ser624, and Tyr652 while position Phe656 participates in cation-π and/or π-π interactions.

FIG. 4 illustrates the evaluation of some interactions between astemizole (structure shown in the upper right hand corner) and modified HERGs. Substitutions with non-native amino acids at positions Thr623, Ser624, Tyr652 and Phe656 with hydroxy threonine and valine at Thr623, alanine and threonine at Ser624, phenylalanine and two fluorinated forms thereof at Tyr652, and two fluorinated forms of phenylalanine at Phe656, indicate that positions 623 and 624 interact via hydrogen bonding, position 652 interacts via cation-π and/or π-π, and position 656 may not be involved in binding or involved via hydrophobic interactions. “NB” refers to “no block”.

FIG. 5 illustrates the evaluation of some interactions between dofetilide (structure shown in the upper right hand corner) and modified HERGs as described in FIG. 4.

FIG. 6 illustrates the evaluation of some interactions between haloperidol (structure shown in the upper right hand corner) and modified HERGs as described in FIG. 4. Little or no hydrogen bonding appears present via position 624.

FIG. 7 illustrates the evaluation of some interactions between risperidone (structure shown in the upper right hand corner) and modified HERGs as described in FIG. 4.

FIG. 8 illustrates the evaluation of some interactions between droperidol (structure shown in the upper right hand corner) and modified HERGs as described in FIG. 4. Effects from positions 623 and 624 appear more prominent.

FIG. 9 illustrates the evaluation of some interactions between pimozide (structure shown in the upper right hand corner) and modified HERGs as described in FIG. 4. An effect via position 656 is present.

FIG. 10 illustrates the evaluation of some interactions between loxapine (structure shown in the upper right hand corner) and modified HERGs as described in FIG. 4.

FIG. 11 illustrates the evaluation of some interactions between amoxapine (structure shown in the upper right hand corner) and modified HERGs as described in FIG. 4.

FIG. 12 illustrates the evaluation of some interactions between imipramine (structure shown in the upper right hand corner) and modified HERGs as described in FIG. 4.

FIG. 13 illustrates the evaluation of some interactions between fluphenazine (structure shown in the upper right hand corner) and modified HERGs as described in FIG. 4.

FIG. 14 illustrates the evaluation of some interactions between triflupromazine (structure shown in the upper right hand corner) and modified HERGs as described in FIG. 4.

FIG. 15 illustrates the evaluation of some interactions between cis-flupenthixol (structure shown in the upper right hand corner) and modified HERGs as described in FIG. 4.

FIG. 16 shows the results of evaluations of multiple compounds with various modified HERG ion channels. The abbreviations used reflect, from left to right, Thr623 to Val, Thr623 to hydroxy Thr, Ser624 to Ala, Ser624 to Thr, Tyr652 to Phe, Phe656 to 3,5-Phe-F₂, and 656Phe to 3,5 Phe-F₂.

### DETAILED DESCRIPTION OF MODES OF PRACTICING THE INVENTION

The present invention provides a method of obtaining highly precise binding and interaction information of ligands or drugs with the HERG ion channel by substitution of native amino acids at critical sites within the transmembrane domains of the ion channel. The information elucidated from these novel experiments allow predictive identification of binding sites in molecules or drugs that contribute to or cause undesirable HERG activity as well as ones that alleviate such activity.

As used herein, the term “HERG” means the human ether-a-go-go related potassium ion channel, which has 6 transmembrane chains. This HERG polypeptide exhibits structural similarities to members of the S4-containing superfamily of ion channels and its behavior can be described by typical gating characteristics, such as sigmoidal time course of activation and C-type inactivation. The sequence of a representative human HERG ion channel is shown as SEQ ID NO:9. The HERG amino acid residue positions described herein are relative to that sequence. As would be evident to the skilled person in the art, however, the invention may be practiced with other HERG sequences with modifications of the residues corresponding to those described herein. Such embodiments are within the scope of the present invention.

As used herein, a Voltage-Gated Ion channel (VGIC) refers to a group of cell membrane channel proteins. These proteins of the VGIC family are ion-selective channel proteins found in a wide range of bacteria, archaea and eukaryotes. Functionally characterized members are specific for K⁺, Na⁺ or Ca²⁺. The K⁺ channels usually consist of homotetrameric structures with each subunit possessing six transmembrane spanners (TMSs). Many voltage-sensitive K⁺ channels function with subunits that modify K⁺ channel gating. Some of these auxiliary subunits, but not those of a HERG channel, are oxidoreductases that coassemble with the tetrameric subunits in the endoplasmic reticulum and remain tightly adherent to the subunit tetramer. High resolution structures of some potassium channels, but not of HERG channels are available (e.g. Jiang et al., Nature (2002) May 30;417(6888):515-22). The high resolution structure of a beta subunit is available (Gulbis et al., Cell (1999) June 25;97(7):943-52).

In eukaryotes, each VGIC family channel type has several subtypes based on pharmacological and electrophysiological data. Thus, there are five types of Ca²⁺ channels (L, N, P, Q and T). There are at least ten types of K⁺ channels, each responding in different ways to different stimuli: voltage-sensitive [Ka, Kv, Kvr, Kvs and Ksr], Ca²⁺-sensitive [BK_{Ca}, IK_{Ca} and SK_{Ca}], and receptor-coupled [K_{M} and K_{ACh}]. There are at least six types of Na⁺ channels (I, II, III, μl, H1 and PN3). Tetrameric channels from both prokaryotic and eukaryotic organisms are known in which each subunit possesses 2 TMSs rather than 6, and these two TMSs are homologous to TMSs 5 and 6 of the six TMS units found in the voltage-sensitive channel proteins. The KcsA of S. lividans is an example of such a 2 TMS channel protein. These channels may include the K_{Na} (Na⁺-activated) and K_{Vol} (cell volume-sensitive) K⁺ channels, as well as distantly related channels such as the Tok1 K⁺ channel of yeast. The TWIK-1 and -2, TREK-1, TRAAK, and TASK-1 and -2 K⁺ channels all exhibit a duplicated 2 TMS unit and may therefore form a homodimeric channel. About 50 of these 4 TMS proteins are encoded in the C. elegans genome. Because of insufficient sequence similarity with proteins of the VGIC family, inward rectifier K⁺ IRK channels (ATP-regulated or G-protein-activated), which possess a P domain and two flanking TMSs, are placed in a distinct family (TC #1.A.2). However, substantial sequence similarity in the P region suggests that they are homologous. The subunits of VGIC family members, when present, frequently play regulatory roles in channel activation/deactivation.

As used herein, the HERG assay includes measurements with a modified HERG ion channel. The modified HERG is preferably expressed in Xenopus oocytes as it is contacted, and interacts, with chemical entities (molecules or compounds) of interest.

The receptophore model, as used herein, is the ensemble of steric and electronic features of a biological target that are necessary to ensure optimal supramolecular interactions with a specific ligand and to trigger (or block) the biological function of the target. Non-limiting examples of binding interactions between HERG and a compound or agent (ligand) that binds HERG include hydrogen-bonding, cation-π, π-π, ion pairing, and hydrophobic interactions.

The QT interval as used herein is the time period it takes for cardiac repolarization as measured on an electrocardiogram. Prolongation of this interval can lead to generation of the life threatening ventricular arrhythmia known as torsades de pointes. Ben-Davies et at. (1993) Lancet 341: 1578. Similarly, the long QT syndrome is an abnormality of cardiac muscle repolarization that predisposes affected individuals to a ventricular arrhythmia that can degenerate into ventricular fibrillation and cause sudden death.

As used herein, the electrocardiogram (hereinafter, “ECG”) is a common test for measuring detailed heart rhythms, waves, and beats.

“Non-native” amino acid or amino acid residue refers to an amino acid other than that normally or naturally found at an amino acid position of a HERG ion channel. The non-native amino acid may be either naturally occurring (one of the 20 recognized natural amino acids as provided in Creighton, Proteins, W.H. Freeman and Co. 1984, pp. 2-53) or an unnatural amino acid. The 20 naturally occurring amino acids are glycine, alanine, valine, leucine, isoleucine, serine, threonine, aspartic acid, asparagine, lysine, glutamic acid, glutamine, arginine, histidine, phenylalanine, cysteine, tryptophan, tyrosine, methionine, and proline. Preferably, however, alanine is not used as the non-native amino acid substitution.

As used herein, an “unnatural amino acid” is any amino acid other than one of the 20 naturally occurring amino acids. Non-limiting examples of unnatural amino acids include hydroxy methionine, norvaline, O-methylserine. crotylglycine, hydroxy leucine, allo-isoleucine, norleucine, α-aminobutyric acid, t-butylalanine, hydroxy glycine, hydroxy serine, F-alanine, hydroxy tyrosine, homotyrosine, 2-F-tyrosine, 3-F-tyrosine, 4-methyl-phenylalanine, 4-methoxy-phenylalanine′3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, hexahydrotyrosine, cyclohexanol-alanine, hydroxyl alanine, hydroxy phenylalanine, hydroxy valine, hydroxy isoleucine and hydroxyl glutamine as well as amino acids with cyclopropyl, cyclobutyl, or cyclopentyl side chains.

Hydroxy tyrosine, hydroxyl alanine, hydroxy phenylalanine, hydroxy valine, hydroxy isoleucine and hydroxyl glutamine (all commonly termed α-hydroxyacids) refer to amino acids in which the NH₂ on the α-carbon is replaced by OH.

Other preferred unnatural amino acids are those with side chains that comprise a five or six membered ring of carbon atoms, optionally heterocyclic, such as those substituted with N, S, or O at one or more positions of the ring. The rings are preferably aromatic and substituted with one or more electron withdrawing groups. Non-limiting examples of preferred electron withdrawing groups are —F, —Cl, —Br, —OH, and —CN. Preferred unnatural amino acids include phenylalanine and tyrosine, each modified to have one or more electron withdrawing group on the aromatic ring. Such groups may participate in interactions based upon hydrogen bonding, cation-π, π-π, and/or hydrophobic interactions.

HERG Structure and Function

The HERG ion channel is a member of the depolarization-activated potassium channel family, which has 6 putative transmembrane spanning domains. This is unusual because the ion channel exhibits rectification like that of the inward-rectifying potassium channels, which only have 2 transmembrane domains. Smith et at. (1996) Nature 379:833, studied HERG channels expressed in mammalian cells and found that this inward rectification arises from a rapid, voltage-dependent inactivation process that reduces conductance at positive voltages. The inactivation gating mechanism of HERG resembles that of C-type inactivation, often considered to be the ‘slow’ inactivation mechanism of other potassium channels. Characteristics of this gating suggested a specific role for this channel in the normal suppression of arrhythmias. The role for HERG in suppressing extra beats might help explain the increased incidence of cardiac sudden death in patients that lack HERG currents, either because they carry a genetic defect or because, for example, they are being treated with class III antiarrhythmics that block HERG channels. Therefore, determination of binding interaction of any drug or compound of this type with the HERG channel would provide information on how to avoid this interaction.

Crystallization is one conventional method for studying three-dimensional protein structures and complexes drug compounds. However, determination of a crystal structure is very time-consuming, and the results are not always precise enough to determine all the possible interactions. In case of membrane proteins (i.e. HERG ion channel), numerous attempts have failed at co-crystallizing the proteins with various known channel blockers in attempts to study the binding site interactions. Additionally, given the dynamic nature of the HERG channel, a static crystal picture may not be in the proper functional context. Lastly, the conformation of the protein under investigation may be altered due to crystal packing forces. The methods described herein provide highly precise interaction and binding data without crystallography. In the absence of atomic-scale structural data for membrane proteins such as that provided by crystallography, these techniques can provide detailed structural information.

To determine which sites on the HERG ion channel to modify using the inventive methods, it is helpful to look at previous studies with the HERG ion channel. For example, conventional mutagenesis studies of the HERG ion channel can provide information on possible binding sites within the transmembrane domains. See Mitcheson et al. (2002) Proc. Natl. Acad. Sci. 97:12329-12333. The inner cavity of the HERG channel may be much larger than any other voltage-gated potassium channel, based on sequence analysis and comparison with the KcsA homology model. Also unlike other voltage-gated potassium channels, the S6 domains of the HERG channels have two aromatic residues that face into the inner cavity. These residues, among others, may bind drugs, leading to the HERG activity. Previously, it has been reported that the binding site of HERG is comprised of amino acids located on the S6 transmembrane domain (Gly648, Tyr652, and Phe656) and pore helix (Thr623 and Val625). See Mitcheson et al. Therefore, these sites are preferred for replacement with non-native amino acids with use of the thus modified HERG as disclosed herein.

Additionally, the present invention provides for the incorporation of non-native amino acids at positions Ser624, Met645, Leu646, Ser649, Ala653, Gly657, Val659, Ser660, Ile663 and Gln664. Preferred substitutions with non-native amino acids at preferred positions of the invention are as follows.

Thr623: valine, isoleucine, hydroxy-threonine, allo-threonine, fluoromethyl threonine, O-methyl threonine, α-aminobutyric acid, serine, and allo-O-methyl threonine.

Ser624: hydroxy-serine, O-methyl serine, α-aminobutyric acid, threonine, and F-alanine.

Val625: leucine, isoleucine, hydroxy valine, norleucine, norvaline, α-aminobutyric acid, and t-butylalanine as well as an amino acid with a cyclopropyl side chain.

Met645: hydroxy methionine, norvaline, O-methylserine, and crotylglycine.

Leu646: valine, isoleucine, hydroxy leucine, allo-isoleucine, norleucine, norvaline, α-aminobutyric acid, and t-butylalanine as well as an amino acid with a cyclopropyl side chain.

Gly648: hydroxyl glycine.

Ser649: hydroxy serine, α-aminobutyric acid, O-methyl serine, and F-alanine.

Tyr652: phenylalanine, hydroxy tyrosine, homotyrosine, 2-F-tyrosine, 3-F-tyrosine, 4-methyl-phenylalanine, 4-methoxy-phenylalanine, 3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, hexahydrotyrosine, and cyclohexanol-alanine, as well as amino acids with cyclopropyl, cyclobutyl, or cyclopentyl side chains.

Ala653: hydroxy alanine, F-alanine, α-aminobutyric acid, and O-methyl serine.

Phe656: hydroxy phenylalanine, 4-methyl-phenylalanine, 4-methoxy-phenylalanine′3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, and cyclohexanol-alanine, as well as amino acids with cyclopropyl, cyclobutyl, or cyclopentyl side chains.

Gly657: hydroxyl glycine.

Val659: leucine, isoleucine, hydroxy valine, norleucine, norvaline, α-aminobutyric acid, and t-butylalanine as well as an amino acid with a cyclopropyl side chain.

Ser660: hydroxy-serine, O-methyl serine, α-aminobutyric acid, and F-alanine.

Ile663: valine, isoleucine, hydroxy isoleucine, allo-isoleucine, norleucine, norvaline, α-aminobutyric acid, and t-butylalanine as well as an amino acid with a cyclopropyl side chain.

Gln664: valine, glutamic acid, 2-amino-5-oxo-hexanoic acid, 2-amino-4-nitro-butyric acid, and hydroxy glutamine.

Generation of Receptophore Model

An accurate receptophore model is built through identification of amino acids involved in the ligand binding site and the probing of the molecular forces involved. First, anon-native amino acid is incorporated into the HERG ion channel using appropriate methods, such as, but not limited to, site directed mutagenesis for substitution with a naturally occurring amino acid and nonsense suppression methodology for an unnatural amino acid. Altered ion channels are preferably expressed heterologously on Xenopus oocyte membranes. Compounds are screened for binding efficacy to the altered channel. Electrophysiological or biochemical assays are used to measure the effects, if any, of non-native amino acid substitutions on ligand binding. Binding data involving the wild-type versus the altered channel are compared to define the molecular forces involved in ligand binding.

The interaction of acetylcholine with the nicotinic acetylcholine receptor has recently been studied in order to develop the receptophore model for the interactions of the nicotinic agonists described in Zhong et al. (1998) Proc. Natl. Acad. Sci. 95:12088-12093. A clear agonist receptophore model of the nicotinic receptor family will emerge after multiple agonist contact points are identified through systematic mapping of the target binding sites using the in vivo nonsense suppression method for unnatural amino acid incorporation. A number of aromatic amino acids have been identified as contributing to the agonist binding site, suggesting that cation-π interactions may be involved in binding the quaternary ammonium group of the agonist, acetylcholine. A compelling correlation has been shown between (i) ab initio quantum mechanical predictions of cation-π binding abilities and (ii) EC₅₀ values for acetylcholine at the receptor for a series of tryptophan derivatives that were incorporated into the receptor by using in vivo nonsense suppression method for unnatural amino acid incorporation. Such a correlation is seen at one, and only one, of the aromatic residues: tryptophan-149 of the α subunit. This finding indicates that, on binding, the cationic, quaternary ammonium group of acetylcholine makes van der Waals contact with the indole side chain of the α tryptophan-149, providing the most precise structural information to date on this receptor. Upon similar systematic probing of other potential steric and electronic interactions at the acetylcholine binding site, a receptophore model will be built for binding and physiological activity of agonists at the nicotinic receptor.

Naturally occurring amino acids are substituted into the HERG ion channel by use of standard mutagenesis techniques known in the art to modify the corresponding native codon to be a non-native codon. Preferably, the introduced non-native codon is one that is readily recognized or preferred by the expression system used to produce the modified HERG.

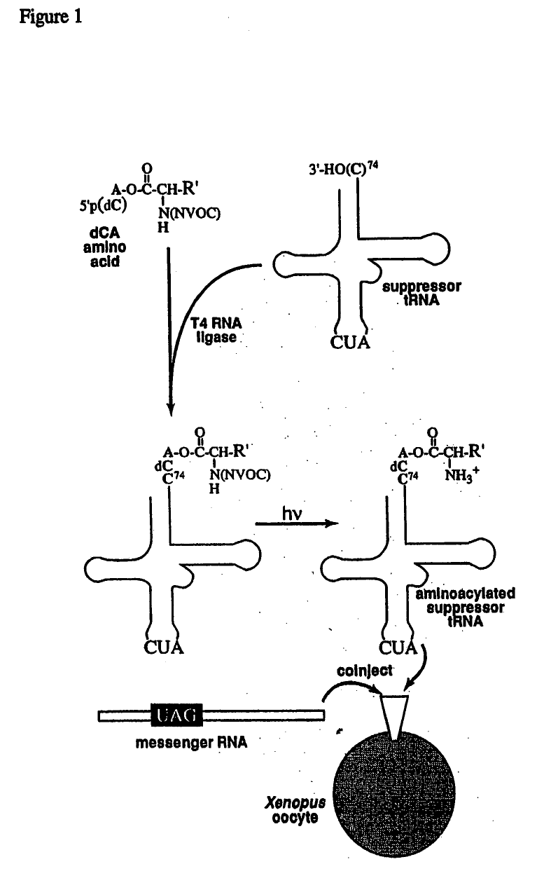

Unnatural amino acids are incorporated into the HERG ion channel binding sites through the use of nonsense codon suppression. Noren et al. (1989) Science 244:182; Nowak et al. (1998) Methods in Enzymol. 293:515. See FIG. 1 herein. In the nonsense suppression method, two RNA species are prepared using standard techniques such as in vitro synthesis from linearized plasmids. The first is an mRNA encoding the HERG channel but engineered to contain an amber stop codon (UAG) at the position where unnatural amino acid incorporation is desired. The second is a suppressor tRNA that contains the corresponding anticodon (CUA) and that is compatible with the expression system employed, such as Tetrahymena thermophila tRNA^{Gln} G73 for Xenopus oocytes or E. coli expression systems. The tRNA is then chemically acylated at the 3′ end with the desired unnatural amino acid using techniques known in the art such as that described in Kearney et al. (1996), Mol. Pharmacol., 50: 1401-1412.

Synthesis of the unnatural amino acids depends on the desired structure. The unnatural amino acid may be prepared, for example, by modification of a natural amino acid. Also, many unnatural amino acids are commercially available.

Additional examples of preferred unnatural amino acids for incorporation into mammalian cells using the methods of the present invention include, but are not limited to, those represented by the following Formula (I): 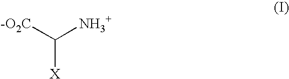
where X is selected from the group consisting of:

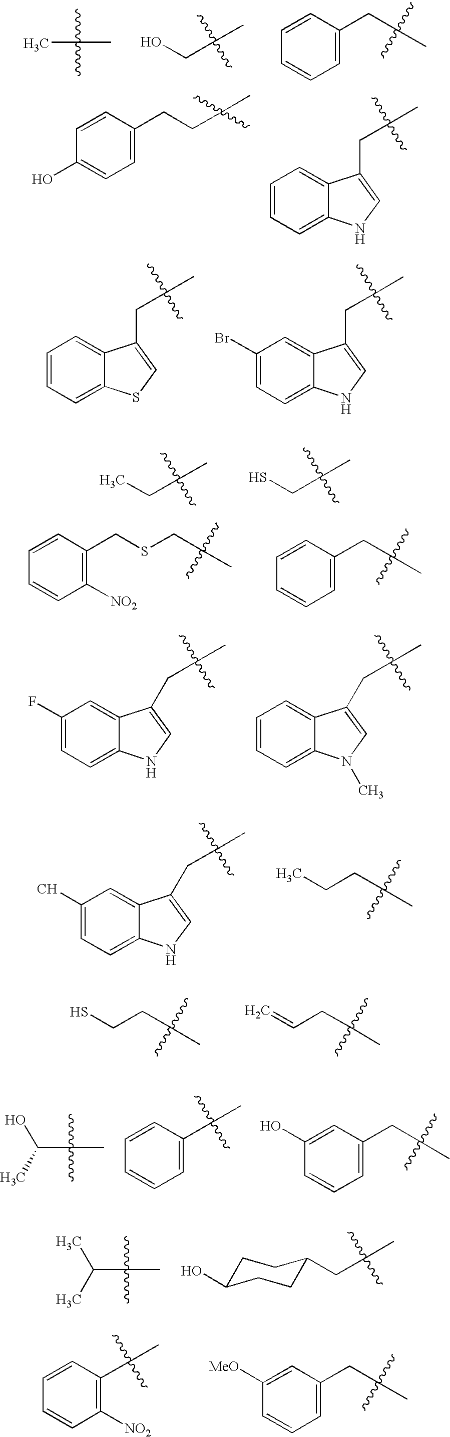

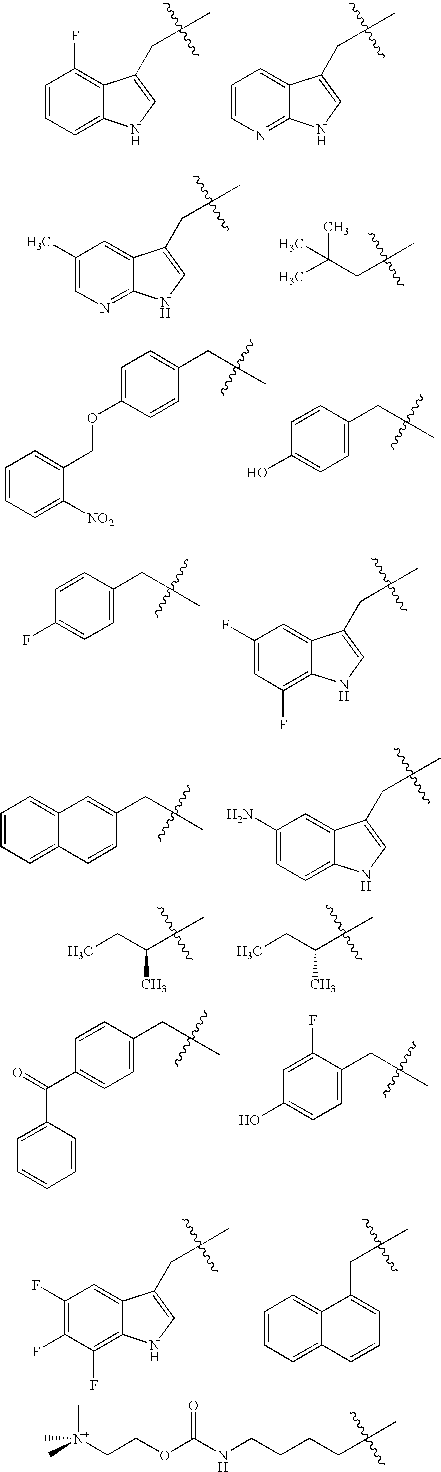

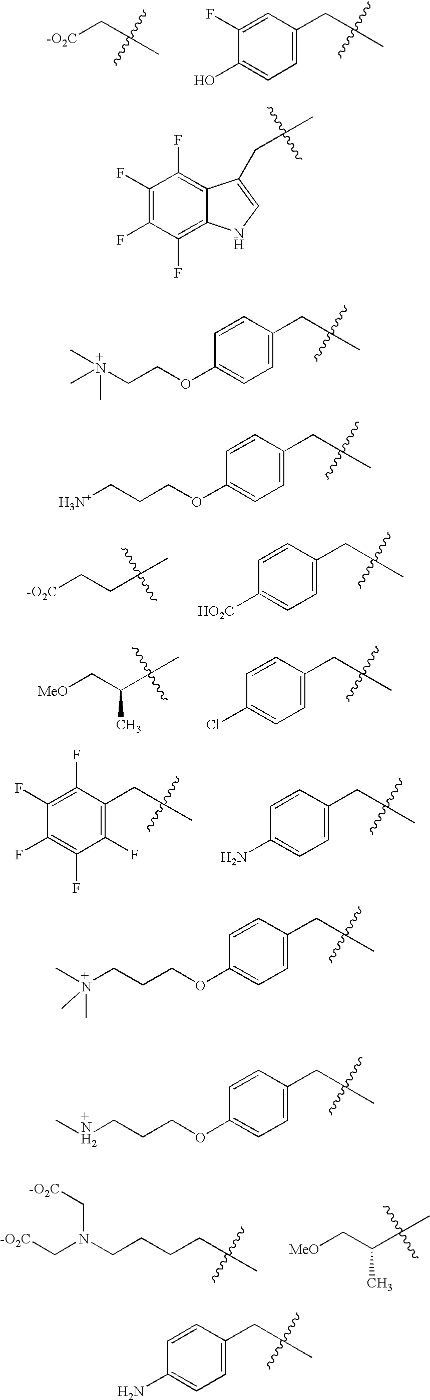

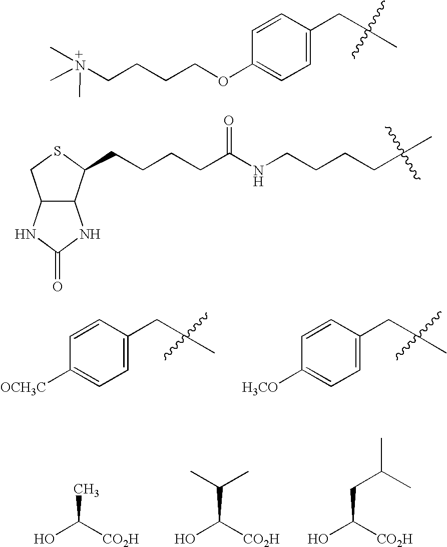

In other preferred embodiments, examples of unnatural amino acids for incorporation into mammalian cells also include, but are not limited to, those represented by the following Formula (II): 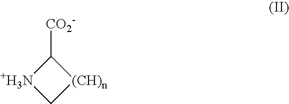
wherein Y is CH₂, (CH)ₙ, N, O, or S, and n is 1 or 2. Examples of such compounds include, but are not limited to, the following compounds:

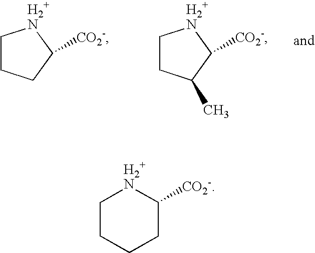

In embodiments comprising an unnatural amino acid, after synthesis of the relevant mRNA and acylated-tRNA, the species are co-injected into intact Xenopus oocytes such as those described in Nowak et al. (1998) Methods in Enzymol 293:515 using standard procedures known in the art. During translation the ribosome incorporates the unnatural amino acid into the nascent peptide at the position of the engineered stop codon, and an altered HERG channel is expressed on the oocyte membrane.

An electrophysiological method such as the current clamp or, preferably, the voltage clamp is used to assess the ligand-binding capabilities of altered ion channels or receptors. The current clamp assay measures ligand binding to a receptor or ion channel by detecting changes in the oocyte membrane potential associated with ion conduction across the cell membrane. These assays may be used to evaluate HERG binding and/or modulation by a molecule or compound of interest.

The voltage clamp measures the voltage-clamp currents associated with ion conduction across the cell membrane. These currents vary with time, with the concentrations of agonists and antagonists, and with membrane potential, and these variations measure the number of open channels at any instant. Such electrophysiological methods are well known in the art (Hille, 2001; Methods in Enzymology, Vol 152) and have been used extensively for the study of ion channels in the Xenopus oocyte expression system.

Other ligand-binding assays can be developed to measure ligand binding events that do not involve changes in membrane potential. While one skilled in the art is capable of selecting a biochemical assay for use with a particular expression system, modified HERG as disclosed herein, ion channel, ligand, and modulator involved in a particular study, we describe here some exemplary ligand-binding assays. The invention is not limited by the particular binding assay employed.

In one embodiment, a labeled ligand is used to physically detect the presence of the bound or unbound ligand. Various types of labels, including but not limited to radioactive, fluorescent, and enzymatic labels, have been used in binding studies and are well known in the art. Labeled ligands can be commercially obtained or prepared using techniques known in the art. A binding assay using a radioactively labeled ligand may include the following steps: (1) incubating purified ion channels or oocytes expressing ion channels with the labeled ligand, (2) allowing an appropriate time for ligand-binding, (3) counting the number of bound ligands using a scintillation counter, and (4) comparing the differences in radioactive counts for altered and unaltered channels.

Ion channel/ligand binding data are compiled to create a model of a ligand binding event. The contribution of specific amino acid side chains to ligand binding is inferred from the comparative properties of a native amino acid with the substituted non-native amino acid. Therefore, the production of meaningful data will depend in part on the selection of appropriate substitutions. While one skilled in the art is capable of selecting a non-native amino acid substitution to investigate a putative channel/ligand interaction, we provide some examples of how relevant information is extrapolated from these experiments.
(1) A cation-π interaction is important if fluoro-, cyano-, and bromo-amino acid derivatives, substituted for natural aromatic amino acids, abrogate ligand binding. When incorporated into an aromatic amino acid, these substituents withdraw electron density from the aromatic ring, weakening the putative electrostatic interaction between a positively charged group on the ligand and the aromatic moiety. Fluoro-derivatives are often preferred because fluorine is a strong electron-withdrawing group, and often adds negligible steric perturbations.
(2) A π-π interaction refers to interaction between aromatic moieties of a weak electrostatic nature, the stabilizing energy of which includes induced dipole and dispersion contributions. There are two general types of aromatic π-π interactions: face-to-face and edge-to-face, wherein the former is usually not of a perfect facial alignment because of the electrostatic repulsion between the two π-systems of the aromatic rings. Instead, the two faces are offset relative to each other and separated by a distance of about 3.3-3.8 Å between the faces. The latter is actually a —C—H to π interaction based on the small dipole moment of the —C—H bond. The attraction in both orientations comes from the interaction between partially positively charged hydrogen atoms on the periphery of the aromatic moiety and the negatively charged π-face of an aromatic system. The importance of a π-π interaction is evident in the same manner as described above for a cation-1 interaction.
(3) Hydrophobic interactions at a given position are important if ligand binding is affected by substitutions that increase hydrophobicity without significantly altering the sterics of the side chain, thereby allowing the importance of hydrophobic interactions to be investigated in the absence of artificial steric constraints. One example of such a manipulation is conversion of a polar oxygen to a nonpolar CH₂ group, as in mutating O-Methyl-threonine to isoleucine. Other methods to increase hydrophobicity, such as increasing side chain length, as in the substitution of allo-isoleucine for valine, or β-branch addition, as in the substitution of norvaline for isoleucine, or γ-branch addition, as in the substitution of t-butylalanine for isoleucine, may produce results that support the importance of hydrophobic interactions. Mutations can be made that also decrease the hydrophobicity of the channel
(4) A local α-helix or β-sheet structure is important if an α-hydroxy acid substitution influences ligand binding. Incorporation of an α-hydroxy acid into the peptide backbone will produce an ester linkage instead of an amide bond. Since the amide hydrogen bond is important for stabilization of local α-helices and β-sheets, the α-hydroxy acid substitution disrupts these structures.
(5) By incorporating the phosphorylated or glycosylated analogue of a given amino acid into the ion channel, the investigator can compare ligand binding in the presence or absence of the putative modification.
(6) Using photoreactive unnatural amino acids, the importance of specific side chains or protein modifications can be studied. For example, addition of the photoremovable nitrobenzyl group to the side chain of an amino acid can prevent interactions with the ligand or block side chain modifications such as phosphorylation and methylation. UV irradiation removes the nitrobenzyl group thereby restoring the amino acid to its native form. Therefore, ligand-binding measurements taken before and after UV irradiation can uncover side chain contributions to ligand binding. Similarly, the importance of local protein structures such as loops can be investigated by incorporating the unnatural amino acid (2-nitrophenyl) glycine (Npg). Irradiation of the Npg-modified amino acid triggers proteolysis of the protein channel backbone. If UV irradiation disrupts ligand binding to the Npg-modified channel, a structure near the incorporated unnatural amino acid is likely important.
(7) Fluorescent reporter groups such as the nitrobenzoxadiazole (NBD) fluorophore or spin labels such as nitroxyl can be incorporated into the ion channel using unnatural amino acids containing these labels. For example, after incorporation of an NBD-amino acid into the channel, fluorescence resonance energy transfer between a fluorescently-labeled ligand and the NBD-amino acid can provide information such as the distance between the amino acid residue and the ligand-binding site.
(8) Electrostatic interactions, such as ion-ion, ion-dipole, and dipole-dipole play an important role in the binding of drugs to proteins. The claimed invention allows us to alter the nature of selected amino acids to test whether these are involved in an electrostatic interaction with a particular drug of interest. For example, we can substitute the neutral amino acid glutamine for the (generally) negatively-charged amino acid glutamate. If this mutation weakens drug binding, we can assume that an electrostatic interaction with the wild-type glutamate is important for binding.

Compounds of interest that will be screened for binding affinity to the modified HERG channels of the invention include, but are not limited to antiarrhythmic drugs. It is known that many structurally diverse compounds block HERG channels, therefore, any of these compounds are candidates for screening with the inventive system. Particular preferred compounds include MK-499, terfenadine, cisapride, and dofetilide. Additional non-limiting examples include astemizole, amperozide, droperidol, risperidone, haloperidol, pimozide, loxapine, amoxapine, imipramine, fluphenazine, triflupromazine, and cis-flupenthixol.

Having now generally described the invention, the same will be more readily understood through reference to the following examples which are provided by way of illustration, and are not intended to be limiting of the present invention, unless specified.

### EXAMPLES

### Example 1

Materials:

DNA oligonucleotides were synthesized on an Expedite DNA synthesizer (Perceptive Biosystems, Framingham, Mass.). Restrictions endonucleases and T4 ligase were purchased from New England Biolabs (Beverly, Mass.). T4 polynucleotide kinase, T4 DNA ligase, and Rnase inhibitor were purchased from Boehringer Mannheim Biochemicals (Indianapolis, Ind.). ³⁵S-methionine and ¹⁴C-labeled protein molecular weight markers were purchased from Amersham (Arlington Heights, Ill.). Inorganic pyrophosphatase is purchased from Sigma (St. Louis, Mo.). Stains-all is purchased from Aldrich (Milwaukee, Wis.). T7 RNA polymerase is either purified using the method of Grodberg and Dunn (1988) J. Bact. 170:1245 from the overproducing strain E. coli BL21 harboring the plasmid pAR1219 or purchased from Ambion (Austin, Tex.). For all buffers described, unless otherwise noted, final adjustment of pH is unnecessary.

Unnatural Amino Acids:

While most unnatural amino acids were purchased from commercial sources, other unnatural amino acids can be synthesized by known techniques. Tryptophan analogues were prepared using the method of Gilchrist et al. (1979) J. Chem. Soc. Chem. Commun. 1089-90. Tetrafluoroindole was prepared by the method of Rajh et al. (1979) Int. J. Pept. Protein Res. 14:68-79. 5, 7-Difluoroindole and 5,6,7-trifluoroindole were prepared by the reaction of CuI/dimethylformamide with the analogous 6-trimethylsilylacetylenylaniline.

Typically, the amino group is protected as the o-nitroveratryloxycarbonyl (NVOC) group, which is subsequently removed photochemically according to methods known in the art. However, for amino acids that have a photoreactive sidechain, an alternative, such as the 4-pentenoyl (4PO) group, a protecting group first described by Fraser-Reid, must be used. Madsen et al. (1995) J. Org. Chem. 60, 7920-7926; Lodder et al. (1997) J. Org. Chem. 62, 778-779. We present here a representative procedure based on the unnatural amino acid (2-nitrophenyl)glycine (Npg), as described in England, et al. Proc. Natl. Acad. Sci. USA (in press).

N-4PO-D,L-(2-nitrophenyl)glycine. The unnatural amino acid D,L-(2-nitrophenylglycine) hydrochloride was prepared according to Davis et al. (1973) J. Med. Chem. 16, 1043-1045; Muralidharan et al. (1995) J. Photochem. Photobiol. B: Biol. 27, 123-137. The amine was protected as the 4-pentenoyl (4PO) derivative as follows. To a room temperature solution of (2-nitrophenyl)glycine hydrochloride (82 mg, 0.35 mmol) in H₂O:dioxane (0.75 ml:0.5 ml) was added Na₂CO₃ (111 mg, 1.05 mmol), followed by a solution of 4-pentenoic anhydride (70.8 mg, 0.39 mmol) in dioxane (0.25 ml). After 3 hours the mixture was poured into saturated NaHSO₄ and extracted with CH₂Cl₂. The organic phase was dried over anhydrous Na₂SO₄ and concentrated in vacuo. The residual oil was purified by flash silica gel column chromatography to yield the title compound (73.2 mg, 75.2%) as a white solid. ¹H NMR (300 MHz, CD₃OD) δ 8.06 (dd, J=1.2, 8.1 Hz, 1H), 7.70 (ddd, J=1.2, 7.5, 7.5 Hz, 1H), 7.62-7.53 (m, 2H), 6.21 (s, 1H), 5.80 (m, 1H), 5.04-4.97 (m, 2H), 2.42-2.28 (m, 4H). HRMS calcd. for Cl₃H₁₄N₂O₅ 279.0981, found 279.0992.

N-4PO-D,L-(2-nitrophenyl)glycinate cyanomethyl ester. The acid was activated as the cyanomethyl ester using standard methods known in the art. (Robertson et al. (1989) Nucleic Acids Res. 17, 9649-9660; Ellman et al. (1991) Meth. Enzym. 202, 301-336. To a room temperature solution of the acid (63.2 mg, 0.23 mmol) in anhydrous DMF (1 ml) was added NEt₃ (95 μl, 0.68 mmol) followed by ClCH₂CN (1 ml). After 16 hours the mixture was diluted with Et₂O, and extracted against H₂O. The organic phase was washed with saturated NaCl, dried over anhydrous Na₂SO₄, and concentrated in vacuo. The residual oil was purified by flash silica gel column chromatography to yield the title compound (62.6 mg, 85.8%) as a yellow solid. ¹H NMR (300 MHz, CDCl₃) δ 8.18 (dd, J=1.2, 8.1 Hz, 1H), 7.74-7.65 (m, 2H), 7.58 (ddd, J=1.8, 7.2, 8.4 Hz, 1H), 6.84 (d, J=7.8 Hz, 1H), 6.17 (d, J=6.2 Hz, 1H), 5.76 (m, 1H), 5.00 (dd, J=1.5, 15.6 Hz, 1H), 4.96 (dd, J=1.5, 9.9 Hz, 1H), 4.79 (d, J=15.6 Hz, 1H), 4.72 (d, J=15.6 Hz, 1H), 2.45-2.25 (m, 4H). HRMS calcd. for Cl₆H₁₇N₃O₅ 317.1012, found 317.1004.

N-4PO-(2-nitrophenyl)glycine-dCA. The dinucleotide dCA was prepared as reported by Schultz (Id.) with the modifications described by Kearney et al. (1996) Mol. Pharmacol. 50, 1401-1412. The cyanomethyl ester was then coupled to dCA as follows. To a room temperature solution of dCA (tetrabutylammonium salt, 20 mg, 16.6 μmol) in anhydrous DMF (400 μl) under argon was added N-4PO-D,L-(2-nitrophenyl)glycinate cyanomethyl ester (16.3 mg, 51.4 μmol). The solution was stirred for 1 hour and then quenched with 25 mM NH₄OAc, pH 4.5 (20 μl). The crude product was purified by reverse-phase semi-preparative HPLC (Whatman Partisil 10 ODS-3 column, 9.4 mm×50 cm), using a gradient from 25 mM NH₄OAc, pH 4.5 to CH₃CN. The appropriate fractions were combined and lyophilized. The resulting solid was redissolved in 10 mM HOAc/CH₃CN and lyophilized to afford 4PO-Npg-dCA (3.9 mg, 8.8%) as a pale yellow solid. ESI-MS M⁻ 896 (31), [M-H]⁻ 895 (100), calcd for C₃₂H₃₆N₁₀O₁₇P₂ 896. The material was quantified by UV absorption (ε₂₆₀˜37,000 M⁻¹ cm⁻¹).

Suppressor tRNA Design and Synthesis:

Suppressor tRNA which encode for the desired unnatural amino acid were made, for example, by the methods taught in Nowak et al. (1998) and Petersson et al. (2002) RNA 8(4):542-7. The following procedure was followed for the suppressor tRNA THG73. The gene for T. thermophila tRNA^{Gln} CUA G73, flanked by an upstream T7 promoter and a downstream Fok I restriction site, and lacking CA at positions 75 and 76, was constructed from eight overlapping DNA oligonucleotides (SEQ ID NOs: 1-8), shown below, and cloned into the pUC19 vector.

SEQ ID NO:1

5′-AATTCGTAATACGACTCACTATAGGTTCTATAG-3′

SEQ ID NO:2

3′- GCATTATGCTGAGTGATATCCAAGA-5′

SEQ ID NO:3

5′-TATAGCGGTTAGTACTGGGGACTCTAAA-3′

SEQ ID NO:4

3′-TATCATATCGCCAATCATGACCCCTGAG-5′

SEQ ID NO:5

5′-TCCCTTGACCTGGGTTCG-3′

SEQ ID NO:6

3′-ATTTAGGGAACTGGACCC-5′

SEQ ID NO:7

5′-AATCCCAGTAGGACCGCCATGAGACCCATCCG -3′

SEQ ID NO:8

3′-AGCTTAGGGTCATCCTGGCGGTACTCTGGGTAGGCCTAG-5′

Digestion of the resulting plasmid (pTHG73) with Fok I gave a linearized DNA template corresponding to the tRNA transcript, minus the CA at positions 75 and 76. In vitro transcription of Fok I linearized pTHG73 was done as described by Sampson et al. (1988) Proc. Natl. Acad. Sci. 85:1033. The 74-nucleotide tRNA transcript, tRNA-THG73 (minus CA), was purified to single nucleotide resolution by denaturing polyacrylamide electrophoresis and then quantitated by ultraviolet absorption.

Chemical Acylation of tRNAs and Removal of Protecting Groups:

The α-NH₂-protected dCA-amino acids or dCA were enzymatically coupled to the THG73 FokI runoff transcripts using T4 RNA ligase to form a full-length chemically charged α-NH₂-protected aminoacyl-THG73 or a full-length but unacylated THG73-dCA.

Prior to ligation, 10 μl of THG73 (1 μg/μl in water) was mixed with 5 μl of 10 mM HEPES, pH 7.5. This tRNA/HEPES premix was heated at 95° C. for 3 min and allowed to cool slowly to 37° C.

After incubation at 37° C. for 2 hours, DEPC-H₂O (52 μl) and 3M sodium acetate, pH 5.0 (8 μl), were added and the reaction mixture was extracted once with an equal volume of phenol (saturated with 300 mM sodium acetate, pH 5.0):CHCl₃: isoamyl alcohol (25:24:1) and once with an equal volume of CHCl₃: isoamyl alcohol (24:1), then precipitated with 2.5 volumes of cold ethanol at −20° C. The mixture was centrifuged at 14,000 rpm at 4° C. for 15 min, and the pellet was washed with cold 70% (v/v) ethanol, dried under vacuum, and resuspended in 7 μl 1 mM sodium acetate, pH 5.0. The amount of α-NH₂— protected amino acyl-THG73 was quantified by measuring A₂₆₀, and the concentration was adjusted to 1 μg/μl with 1 mM sodium acetate pH 5.0.

The ligation efficiency was determined from analytical PAGE. The α-NH₂-protected amino acyl-tRNA partially hydrolyzes under typical gel conditions, leading to multiple bands, so the ligated tRNA was deprotected prior to loading. Such deprotected tRNAs immediately hydrolyze on loading. Typically, 1 g of ligated tRNA in 10 μl BPB/XC buffer was loaded onto the gel, and 1 μg of unligated tRNA was run as a size standard. The ligation efficiency was determined from the relative intensities of the bands corresponding to ligated tRNA (76 bases) and unligated tRNA (74 bases).

Generation of mRNA:

For introduction of an unnatural amino acid, the mRNA was synthesized in vitro from a mutated complementary cDNA clone containing a stop codon, TAG, at the amino acid position of interest (the amino acid position in which an unnatural amino acid would be substituted into HERG) for use in the nonsense codon method. For introduction of a non-native, but naturally occurring amino acid, a codon for the non-native amino acid is introduced. Use of codons preferentially used in eukaryotic cells, such as Xenopus oocytes and mammalian cells, are preferred in the practice of the invention.

In the nonsense codon suppression method, it is desirable to have the gene of interest in a high-expression plasmid, so that functional responses in oocytes may be observed 1-2 days after injection. Among other considerations, this minimizes the likelihood of reacylation of the suppressor tRNA. Although there are many high-expression oocyte plasmids available to one of skill in the art, we describe here the high-expression plasmid pAMV-PA, generated by modifying the multiple cloning region of pBluescript SK+. Nowak et al. (1998) Methods in Enzymol. 293:515.

At the 5′ end, an alfalfa mosaic virus (AMV) sequence was inserted, and at the 3′ end a poly(A) tail was added, providing the plasmid pAMV-PA. mRNA transcripts containing the AMV region bind the ribosomal complex with high affinity, leading to 30 fold increase in protein synthesis. Including a 3′poly (A) tail was shown to increase mRNA half-life, therefore increasing the amount of protein synthesized. The gene of interest was subcloned into pAMV-PA such that the AMV region is immediately 5′ of the ATG start codon of the gene (i.e. the 5′ untranslated region of the gene was completely removed). The plasmid pAMV-PA was made available from C. Labaraca at Caltech.

TAG stop codons at positions where unnatural amino acid incorporation is desired were produced by site directed mutagenesis. Suitable site-directed mutagenesis methods used to create stop codons at the desired positions include the Transformer kit (Clontech, Palo Alto, Calif.), the Altered Sites kit (Stratagene, La Jolla, Calif.), and standard polymerase chain reaction (PCR) cassette mutagenesis procedures. With the first two methods, a small region of the mutant plasmid (400-600 base pairs) was subcloned into the original plasmid. The same methods may be used to introduce a codon for a non-native amino acid at positions where mutagenesis is desired. With all methods, the inserted DNA regions were checked by automated sequencing over the ligated sites. The pAMV-PA plasmid constructs were linearized with NotI, and mRNA transcripts were generated using the mMessage mMachine T7 RNA polymerase kit (Ambion, Austin, Tex.).

Oocytes—Preparation and Injection:

Oocytes were removed from Xenopus laevis using techniques known in the art. Quick, M., Lester, H. A. (1994). Methods for expression of excitability proteins in Xenopus Oocytes. In Ion Channels of Excitable Cells. (Narahashi, T., ed.), pp 261-279, Academic Press, San Diego, Calif., USA. Oocytes were maintained at 18° C. in ND96 solution consisting of 96 mM NaCl, 2 mM KCl, 1 mM MgCl₂, 1.8 mM CaCl₂, and 5 mM HEPES (pH 7.5), supplemented with sodium pyruvate (2.5 mM), gentamicin (50 μg/ml), theophylline (0.6 mM) and horse serum (5%). Prior to injection, the NVOC-aminoacyl-tRNA (1 μg/μl) in 1 mM NaOAc (pH 5.0) was deprotected by irradiating for 5 min with a 1000 W xenon arc lamp (Oriel) operating at 600 W equipped with WG-335 and UG-11 filters (Schott). The deprotected aminoacyl-tRNA was mixed 1:1 with a water solution of the desired mRNA. Oocytes were injected with 50 nl of a mixture containing 25-50 ng aminoacyl-tRNA and 12.5-18 ng of total ion channel mRNA (ratio of 20:1:1:1 for α:β:γ:δ subunits). In the case of substitutions with non-native, but naturally occurring amino acids, no tRNA need be injected.

Electrophysiology:

Two-electrode voltage-clamp recordings were performed 24 to 36 hours after injection using a GeneClamp500 circuit and a Digidata 1200 digitizer from Axon Instruments, Inc. (Foster City, Calif.) interfaced with a PC running pCLAMP6 or CLAMPEX software from Axon. The recording solution contained 96 mM NaCl, 2 mM MgCl₂, and 5 mM HEPES (pH 7.4). Whole-cell current responses to various ligand concentrations at indicated holding potentials (typically −60 mV) were fitted to the Hill equation, I/Iₘₐₓ=1/{1+(EC₅₀/[A])ⁿ}, where I is agonist-induced current at [A], Iₘₐₓ is the maximum current, EC₅₀ is the concentration inducing half-maximum response, and n is the Hill coefficient.

Development of Receptophore Model:

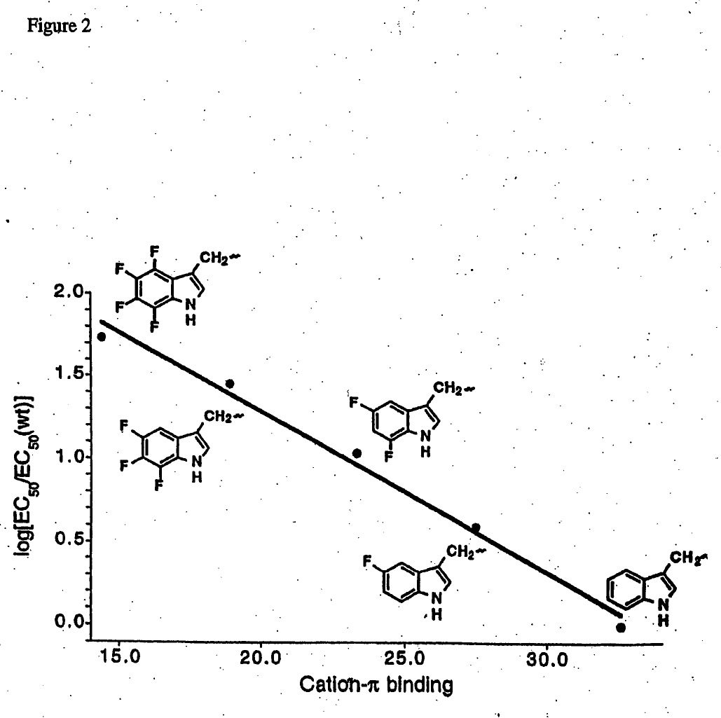

Dose-response curves were fitted with the Hill equation for the unaltered receptor (WT) and for unnatural amino acid substitutions at α-Trp 149. Substitutions include 5-F-Trp, 5,7-F₂-Trp, 5,6,7-F₃-Trp, and 4,5,6,7-F₄-Trp. The log[EC₅₀/EC₅₀ (WT)] for each substitution and for the unaltered receptor was plotted vs. cation-π binding ability of each fluorinated Trp derivative. Cation-π binding ability for both trp and the fluorinated derivatives was predicted using ab initio quantum mechanical calculations. Mecozzi et al. (1996) J. Amer. Chem. Soc. 118: 2307-2308; Mecozzi et al. (1996) Proc. Natl. Acad. Sci. USA 93:10566-10571. Data fit the line y=3.2-0.096×, with a correlation coefficient r=0.99. See FIG. 2. These data are consistent with a cation-π bond between α-Trp 149 and the quaternary ammonium of acetylcholine in the bound position because each substitution's EC₅₀ value corresponds well with the predicted loss in binding energy due to the substitution. After further systematic mapping of contacts between acetylcholine and the nicotinic acetylcholine receptor, a receptophore model describing the complete steric and electronic features involved in this interaction can be made.

### Example 2

Characterization of the Cation-π Interaction Site at Y652 and F656 Using Dofetilide:

The results of the binding and electrophysiology studies of dofetilide and several of its analogues with the HERG channel and several of its mutants containing unnatural amino acid mutations at the Y652 and F656 sites were used to generate a detailed picture of the binding at this site. The dofetilide analogues are chosen to represent a range of binding affinities to the HERG channel. This approach provides a range of interactions that allow for the definition of the pharmacophore for dofetilide binding to the HERG channel. The unnatural HERG channel mutants reveal details of the binding interactions that provide indications of the orientations of dofetilide and its analogues at the binding site. The dofetilide and dofetilide analogues used in this experiment, shown below, are known in the art and described in, for example, U.S. Pat. No. 4,959,366 and EP 649,838.

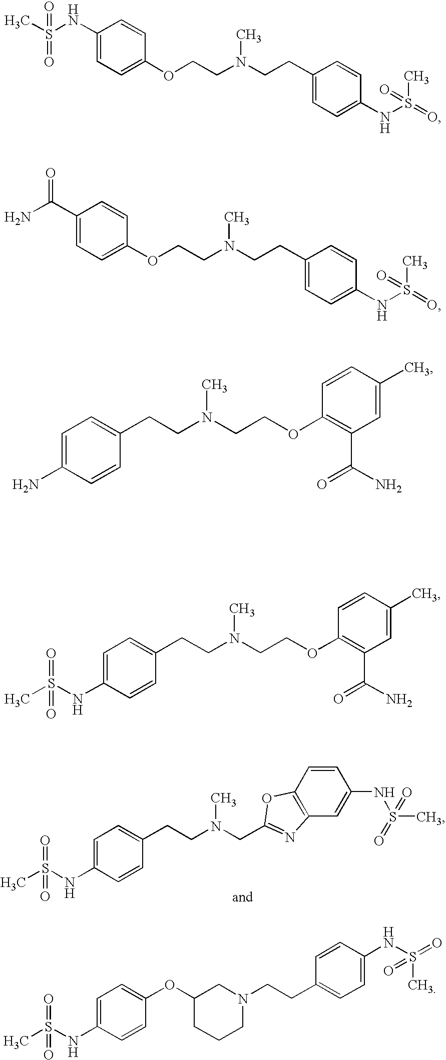

### Example 3

Interactions Between HERG Ion Channel and Various Molecules:

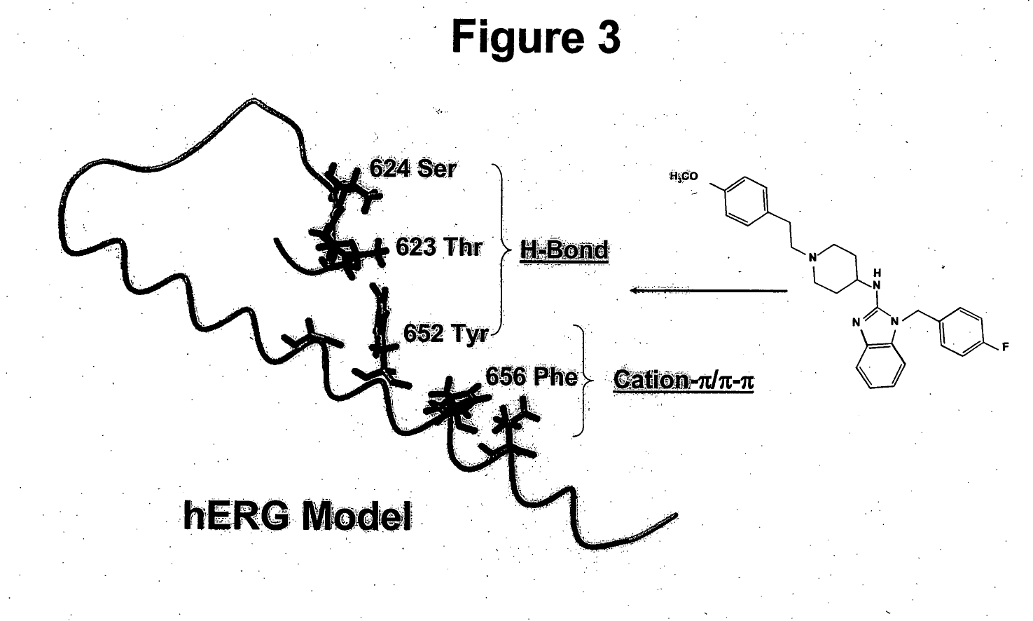

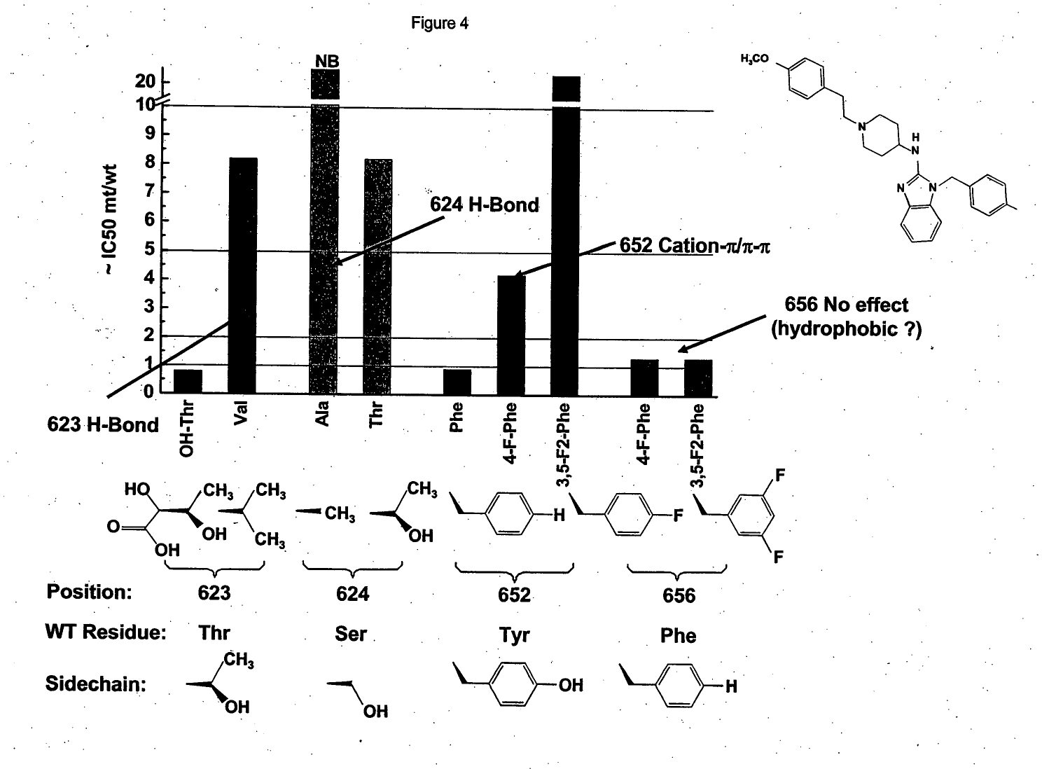

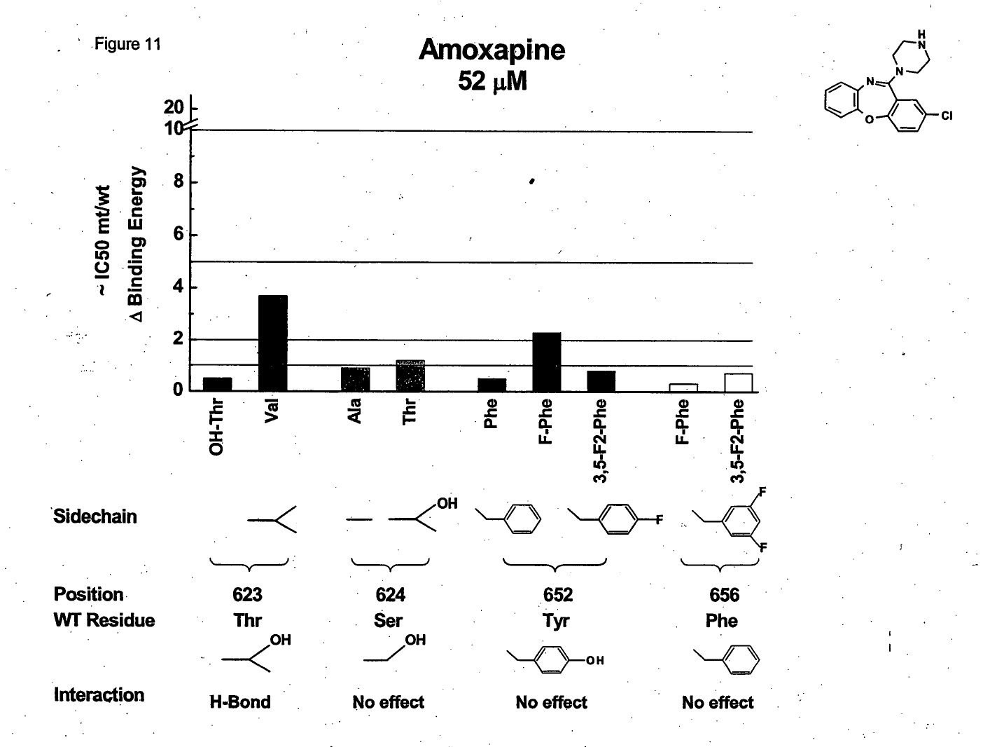

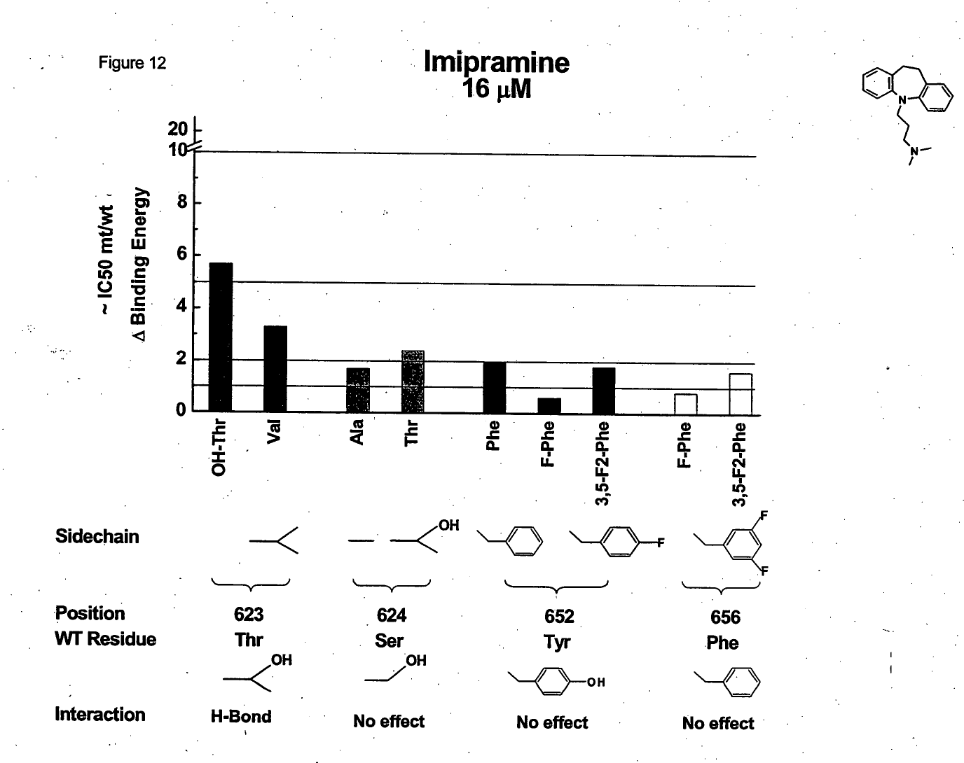

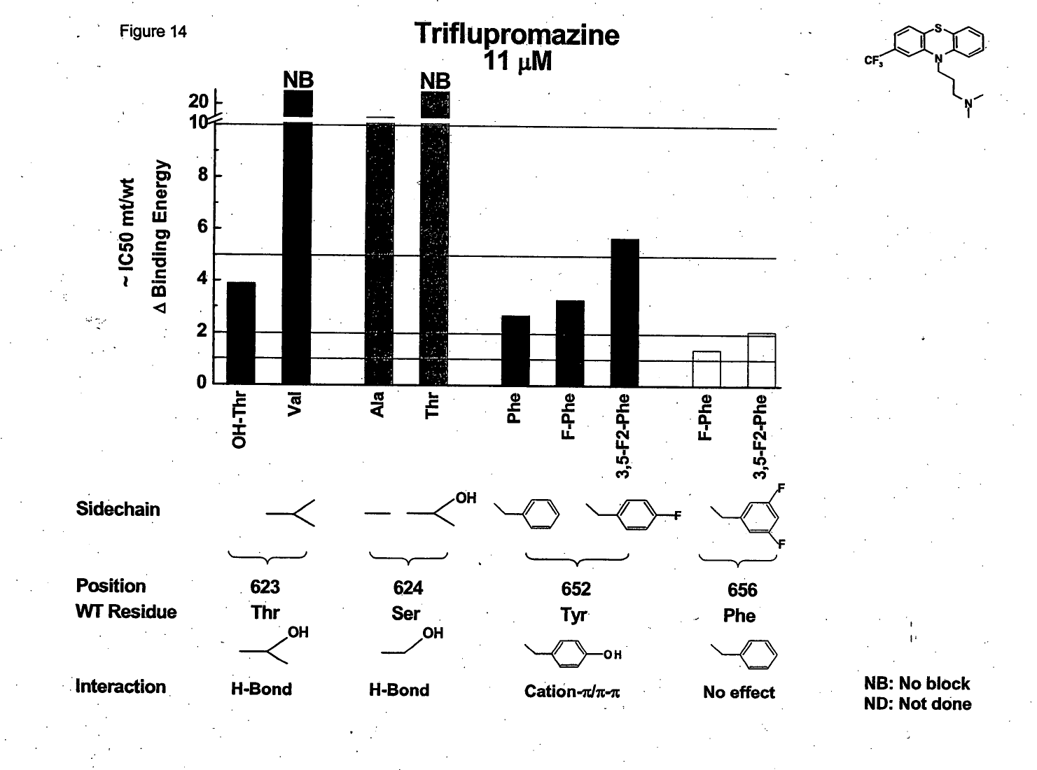

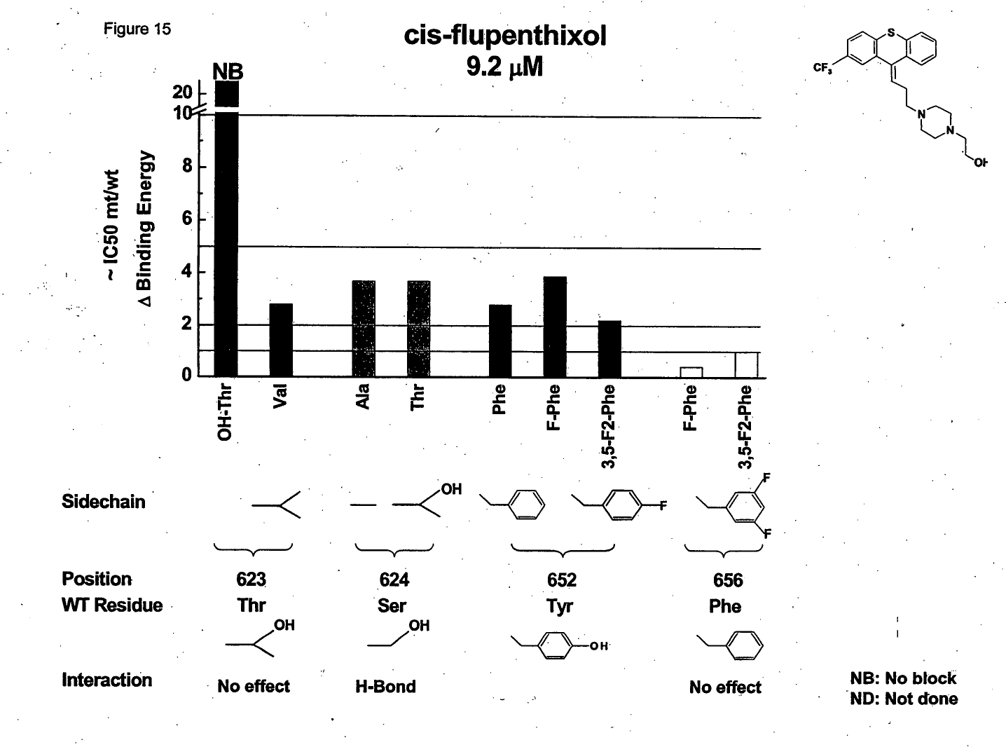

The possible relevance of positions Thr623, Ser624, Tyr652 and Phe656 of HERG is illustrated in FIG. 3. Modified HERG channels comprising individual substitutions at each of these four positions were prepared as described herein. The interaction of these modified HERG channels and various known HERG blocking drugs was evaluated and the results shown in FIGS. 4-15.

FIGS. 4-6 show the results with astemizole, dofetilide, haloperidol, and pimozide, respectively. With respect to FIG. 4, substitutions with unnatural amino acids at positions Tyr652 and Phe656 with two fluorinated forms of phenylalanine at each indicate that position 652 is involved in a cation-π and/or π-π interaction, based on the increase in the IC₅₀ ratio with the doubly fluorinated phenylalanine relative to the singly fluorinated phenylalanine and phenylalanine alone, and position 656 may not be involved in binding or involved via hydrophobic interactions because the two fluorinated phenylalanines gave the same results.

The results of substitution with hydroxy threonine and valine at position 623 indicates that the —OH moiety of threonine participates in interactions between HERG and astemizole. The loss of blocking activity with the substitution of alanine at position 624 indicates that the —OH moiety of serine participates in interactions between HERG and astemizole.

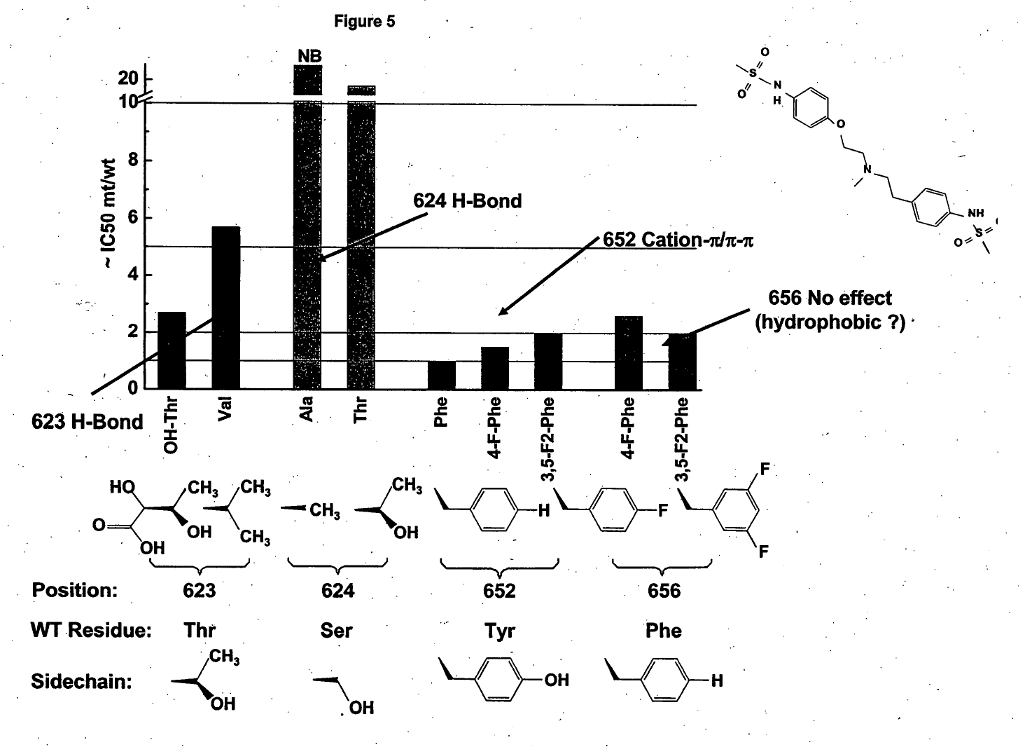

FIG. 5 shows the results of the same substitutions in HERG when dofetilide is used. The results with the singly and doubly fluorinated, as well as naturally occurring, phenylalanine indicate that position 652 interacts via cation-π and/or π-π, while position 656 may not be involved in binding or involved via hydrophobic interactions because of the relative results with the two fluorinated phenylalanine substitutions. The results at positions 623 and 624 are analogous to those discussed above for astemizole.

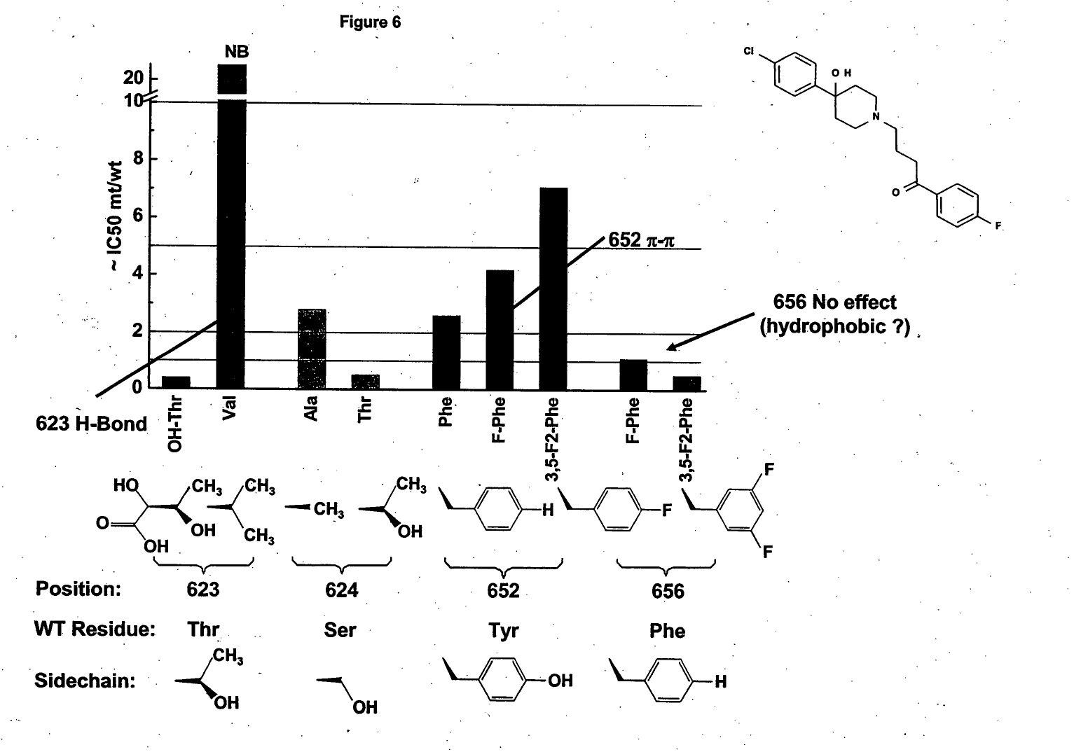

FIG. 6 shows the results of the same substitutions in HERG when haloperidol is used. The results for positions 623, 652, and 656 are analogous to that discussed for dofetilide above. The results at position 624 suggests that the serine there might not participate in hydrogen bonding.

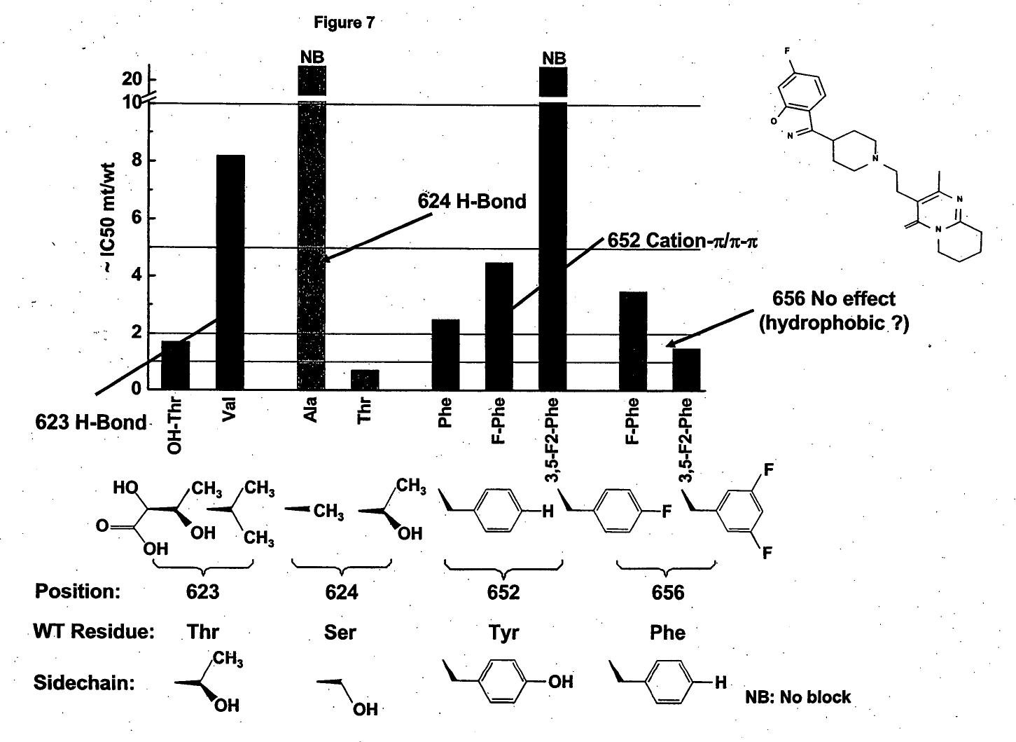

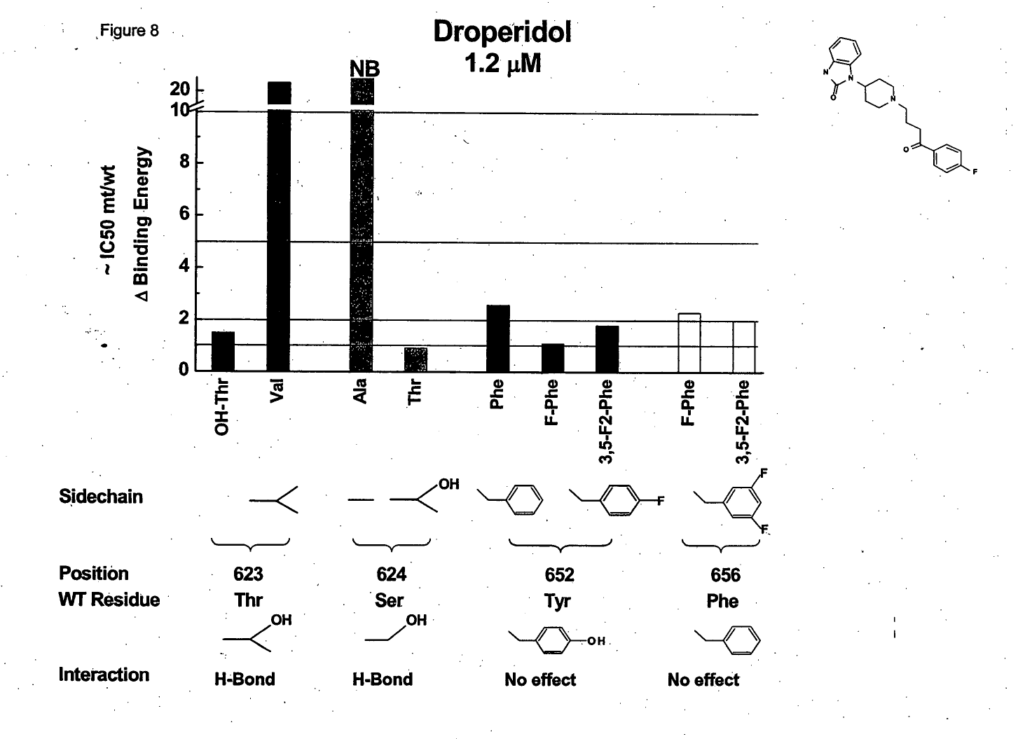

FIGS. 7 and 8 show the results with risperidone and droperidol, respectively. These two compounds are structurally similar.

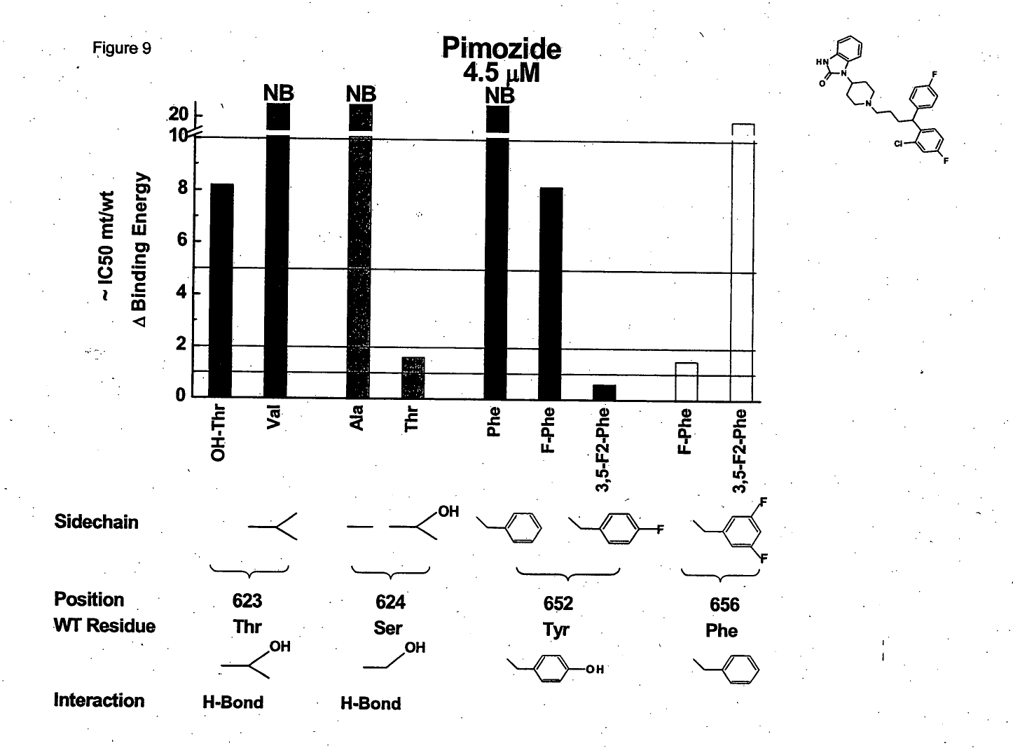

FIG. 9 shows the results with pimozide, which are similar to those of FIGS. 4-6 above except that position 656 is likely involved in cation-π and/or π-π interactions.

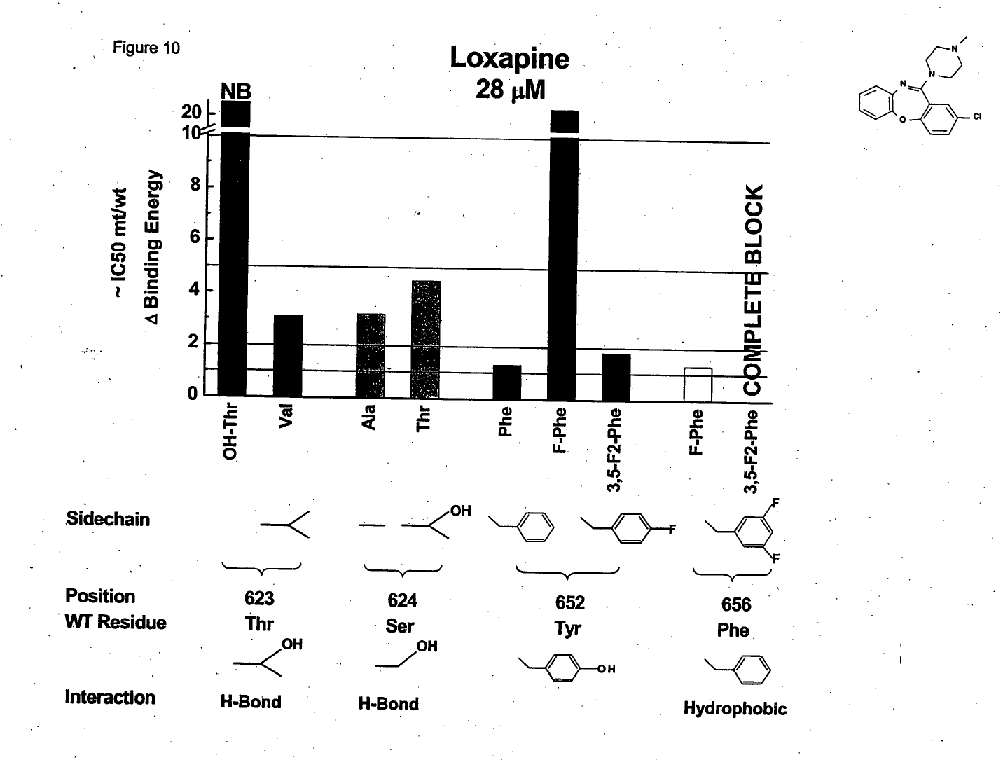

FIGS. 10-12 show the results with loxapine, amoxapine, and imipramine, respectively. These results demonstrate how structurally similar compounds may not have the same interactions with HERG. This highlights one of the benefits provided by the instant invention in providing valuable information where structurally similar HERG blockers display distinct binding modes.

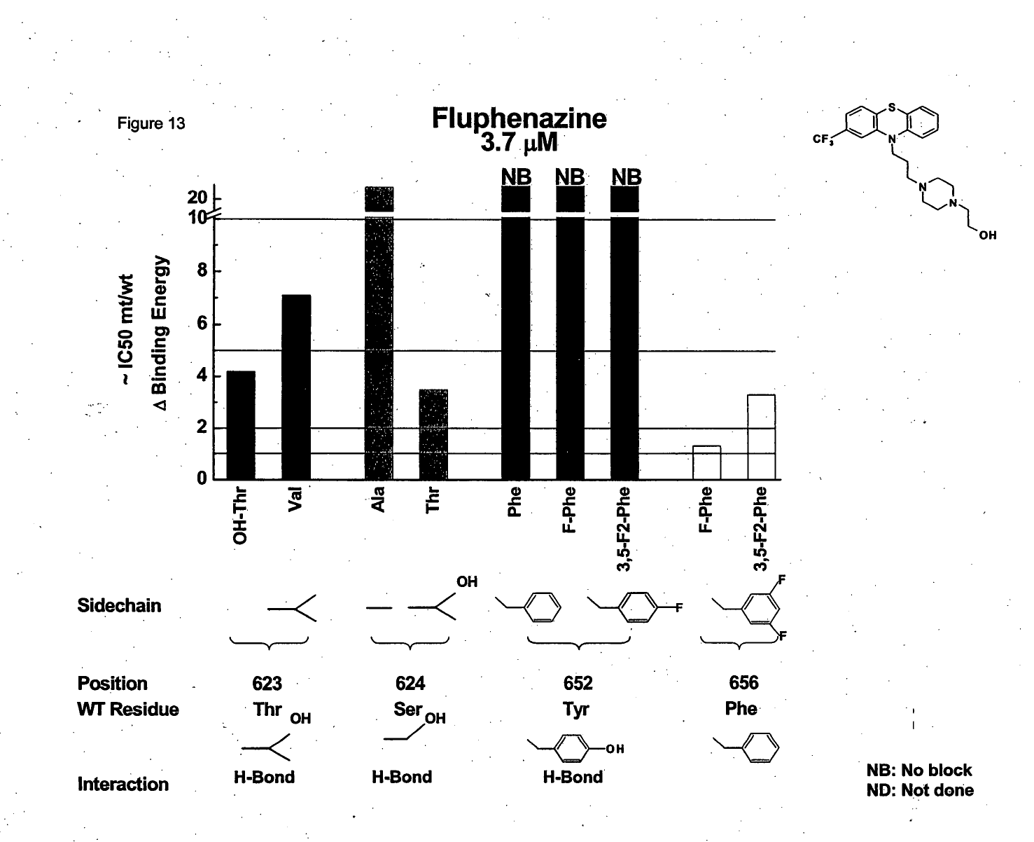

FIGS. 13-15 show the results with fluphenazine, triflupromazine, and cis-flupenthixol, respectively. Like the case with FIGS. 10-12, these three compounds are structurally similar but apparently quite distinct in how they interact with HERG.

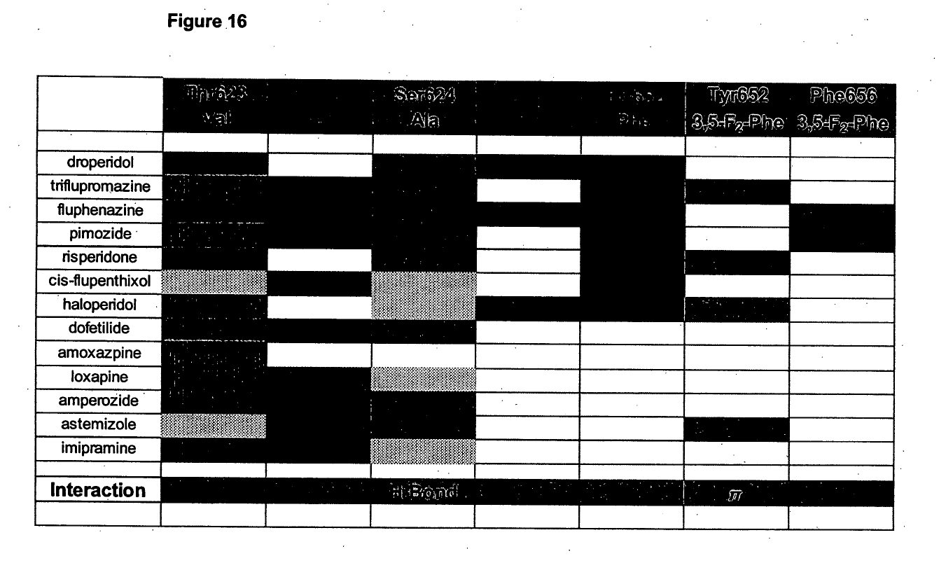

FIG. 16 shows the results for various compounds with modified HERG ion channels. The results reveal specific interactions for known HERG blockers, such as a subset of compounds interact with Tyr652 via cation-π/π-π, interactions; a majority of compounds interact with 623Thr and 624Ser via a hydrogen bond; and a majority of compounds may interact with Phe656 via a hydrophobic interaction.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A modified HERG ion channel comprising the protein specified by the amino acids in SEQ ID NO:9 and a non-native, naturally occurring amino acid residue substituted at a position selected from
Thr623, Ser624, Val625, Met645, Leu646, Gly648, Ser649, Tyr652, Ala653, Phe656, Gly657, Val659, Ser660, Ile663, and Gln664
wherein said naturally occurring amino acid residue is not alanine.

2. The modified HERG ion channel of claim 1 wherein said non-native amino acid residue is at Thr623.

3. The modified HERG ion channel of claim 1 wherein said non-native amino acid residue is at Ser624.

4. The modified HERG ion channel of claim 1 wherein said non-native amino acid residue is at Tyr652.

5. The modified HERG ion channel of claim 1 wherein said non-native amino acid residue is at Phe656.

6. The modified HERG ion channel of claim 1 wherein said non-native amino acid residue is at Ser660.

7. The modified HERG ion channel according to claim 1 expressed in vivo.

8. The modified HERG ion channel according to claim 7, expressed in a Xenopus oocyte.

9. The modified HERG ion channel according to claim 7, expressed in a mammalian cell.

10. A method of determining the nature of a compound's interaction with HERG comprising
contacting said compound with the modified HERG ion channel of claim 1,
measuring the compound's ability to bind to the altered HERG, and
comparing the results to the same compound's ability to bind to an unmodified HERG or a second modified HERG.

11. The method of claim 10 wherein said second modified HERG comprises an unnatural substitution.

12. The method of claim 11 wherein said unnatural substitution is at the same position as the non-native amino acid of said modified HERG.

13. The method of claim 10 wherein said second modified HERG comprises a non-native, naturally occurring amino acid substitution.

14. The method of claim 13 wherein said substitution is at the same position as the non-native amino acid of said modified HERG.

15. The method of claim 10 wherein said modified HERG and said unmodified HERG or second modified HERG are expressed in Xenopus oocytes.

16. The method of claim 10 wherein said modified HERG and said unmodified HERG or second modified HERG are expressed in mammalian cells.

17. The method of claim 10 further comprising measuring the inhibition of HERG mediated ion conduction across a cell membrane.

18. The method of claim 10 wherein said compound has or may have cardiac toxicity.

19. The method of claim 17 wherein said compound causes or may cause cardiac arrhythmia, ventricular fibrillation, QT interval prolongation, and/or sudden death.

20. A method of determining the nature of a compound's interaction with HERG comprising
contacting said compound with the modified HERG ion channel of claim 1,
measuring the compound's ability to bind to the altered HERG, and
comparing the results to a similar compound's ability to bind to the same modified HERG.
